(19) United States
(12) Patent Application Publication
(10) Pub. No.: US 2014/0271820 A1
(43) Pub. Date: Sep. 18, 2014

(54) LIPOSOME OXALIPLATIN COMPOSITIONS FOR CANCER THERAPY

(71) Applicant: Mallinckrodt LLC, Hazelwood, MO (US)

(72) Inventor: William McGhee, Fenton, MO (US)

(73) Assignee: Mallinckrodt LLC, Hazelwood, MO (US)

(21) Appl. No.: 14/207,260

(22) Filed: Mar. 12, 2014

Related U.S. Application Data
(60) Provisional application No. 61/780,000, filed on Mar. 13, 2013.

(51) Int. Cl. A61K 47/28; A61K 31/282; A61K 47/24; A61K 9/127

(52) U.S. Cl. CPC A61K 47/28; A61K 9/127; A61K 31/282; A61K 9/1277; A61K 47/24
USPC 424/450; 514/492; 264/4.1

(57) ABSTRACT

The present invention provides a composition for the treatment of cancer including zwitterionic liposomes consisting essentially of: 50-70 mol % of a phosphatidylcholine lipid, 25-45 mol % of cholesterol, and 2-8 mol % of a PEG-lipid; and oxaliplatin. Oxaliplatin is encapsulated in the liposomes in an amount such that the ratio of the total lipid weight to the oxaliplatin weight is from about 20:1 to about 65:1. Methods for the preparation of liposomal oxaliplatin and the treatment of cancer are also disclosed.

### CROSS-REFERENCES TO RELATED APPLICATIONS

This application claims the benefit of priority to U.S. Provisional Application Ser. No. 61/780,000, filed Mar. 13, 2013, the content of which is incorporated herein by reference in its entirety.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Platinum-based drugs (or “platins”) are effective anticancer drugs, forming DNA adducts that block DNA and RNA synthesis in cancer cells and inducing apoptosis. Cisplatin, carboplatin, and oxaliplatin are the main platins used for treating numerous solid tumors including ovarian, lung, colorectal, testicular, bladder, gastric, melanoma, and head and neck cancers. However, a major disadvantage of the platins is toxicity. Common side effects include kidney and nerve damage, high-end hearing loss, prolonged nausea, and vomiting. Cisplatin in particular has a very short half-life in the blood which results in acute nephrotoxicity due to excretion of the drug by the kidney.

Oxaliplatin is a platinum-based chemotherapeutic agent with a 1,2-diaminocyclohexane (DACH) carrier ligand. Oxaliplatin differs from cisplatin in that the amine groups of cisplatin are replaced by diaminocyclohexane (DACH) and the two chlorides are replaced by a bidentate oxalate moiety. The molecular weight of oxaliplatin is 397.3 g/mol. The chemical structures of oxaliplatin (I) and cisplatin (II) are shown below.

[chemical structure]

Oxaliplatin has shown in vitro and in vivo efficacy against many tumor cell lines. Although the mechanism of action of oxaliplatin is not completely elucidated, it has been shown that the aqua-derivatives resulting from the biotransformation of oxaliplatin interact with DNA to form both inter- and intra-strand cross links, resulting in the disruption of DNA synthesis leading to cytotoxic and antitumour effects (Raymond, et. al. Annals of Oncology. 9: 1053-1071. 1998). The retention of the bulky DACH ring by activated oxaliplatin is thought to result in the formation of platinum-DNA adducts, which appear to be more effective at blocking DNA replication and are more cytotoxic than adducts formed from cisplatin. Oxaliplatin is especially important in treating against cancers that have exhibited resistance against first-line treatment with either cisplatin or carboplatin (Boulikas & Vougiouka. Oncology Reports. 10: 1663-1682. 2003). No nephrotoxicity has been observed, in contrast to cisplatin, and no hydration is needed during its administration. Kidney tubular necrosis has been rarely observed. Studies also demonstrate additive and/or synergistic activity with a number of other compounds, suggesting the possible use of oxaliplatin in combination therapies such as in combination with fluorouracil both in vitro and in vivo. (Ibrahim, A., et al. The Oncologist. 9: 8-12. 2004).

Unlike cisplatin, oxaliplatin in plasma rapidly undergoes non-enzymatic transformation into reactive compounds because of displacement of the oxalate group, a process that complicates its pharmacokinetic profile. Most of the compounds appear to be pharmacologically inactive, but dichloro(DACH) platinum complexes enter the cell, where they have cytotoxic properties. Although oxaliplatin has shown a wide antitumor effect in vitro and in vivo and a better safety profile than cisplatin, the main adverse reactions are neurotoxicity and hematological and gastrointestinal (GI) toxicity (Ibrahim, et al.).

Liposomes have been used as delivery vehicle for platins in an attempt to reduce the drugs' toxicity. A liposome is a vesicle including a phospholipid bilayer separating exterior and interior aqueous phases. Liposomes are capable of carrying both hydrophobic drugs in the lipid bilayer and/or hydrophilic drugs in the aqueous core for drug delivery. Liposome size typically ranges from 50 to 250 nm in diameter, with diameters of 50 to 150 nm being particular preferable for certain applications. The use of liposomal platins, including oxaliplatin, has presented considerable challenges. Liposomal platins demonstrate unique patterns of distribution, metabolism, and excretion from the body compared with the free drugs, as well as varying toxicity levels and unique side effects. In particular, optimizing the release rate of liposomal platins is a difficult balancing act between in vivo half life and release, or between safety and efficacy. In general, leaky liposomes will make the encapsulated drugs more available, but cause more risk in toxicity similar to the native drugs. On the other hand, less leaky liposomes may reduce toxicity, but they may not provide sufficient drug release for adequate efficacy. Such challenges were reflected in the limited in vivo efficacy of sterically-stabilized liposomal cisplatin (SPI-77) in phase II study trials (Feng, et al. Cancer Chemother. Pharmacol. 54: 441-448. 2004).

Therefore, it is desirable to develop liposomal oxaliplatin with improved properties compared to existing liposomal and non-liposomal platin therapeutics. There is a need for formulations that balance efficacy and safety and improve the bioavailability of oxaliplatin to targeted cancer cells. The present invention addresses these and other needs.

### BRIEF SUMMARY OF THE INVENTION

In one aspect, the invention provides a composition for the treatment of cancer. The composition includes: (a) zwitterionic liposomes consisting essentially of 50-70 mol % of a phosphatidylcholine lipid or mixture of phosphatidylcholine lipids, 25-45 mol % of cholesterol, and 2-8 mol % of a PEG-lipid; and (b) oxaliplatin, encapsulated in the liposomes in an amount such that the ratio of the total lipid weight to the oxaplatin weight is from about 20:1 to about 65:1.

In a second aspect, the invention provides a method of treating cancer. The method includes administering to a subject in need thereof a composition of the invention.

### BRIEF DESCRIPTION OF THE DRAWINGS

1/11

FIG. 1 shows the in vitro release of oxaliplatin from liposomes with varying lipid content.

2/11

FIG. 2 shows the in vitro release of cisplatin from liposomes containing distearoylphosphatidylcholine (A) or palmitoyloleoylphosphatidylcholine (B,C) at pH=7.4 and pH=5.

FIG. 3 shows the in vitro release of oxaliplatin from liposomes containing palmitoyloleoylphosphatidylcholine at pH=7.4 and pH=5.

FIG. 4 shows the release rate of oxaliplatin from POPC/Chol/DSPE-PEG2000 liposomes in PBS (pH 7.4 and 5) and FBS.

4/11

FIG. 5 shows the correlation of % oxaliplatin release to mole % cholesterol in POPC/Chol/DSPE-PEG2000 liposomes

FIG. 6 shows the correlation of IC₅₀ to mole % cholesterol in POPC/Chol/DSPE-PEG2000 liposomes.

5/11

FIG. 7 shows the correlation of IC₅₀ to oxaliplatin release rate in POPC/Chol/DSPE-PEG2000 liposomes.

FIG. 8 shows mean KB tumor volume measured after a single intravenous administration of liposomal oxaliplatin (liposomal oxaliplatin 5a) at 40 and 60 mg/kg, free oxaliplatin at 15 mg/kg (MTD), or saline (control). Liposomal oxaliplatin 5a at both doses tested significantly inhibited tumor growth compared to oxaliplatin at day 27 post dose (#, P<0.05) and control on day 31 post dose (*, P<0.05). One-way ANOVA followed by Neuman-Keuls post hoc test. Values are mean±SEM for 5-10 mice/group.

6/11

FIG. 9 shows a Kaplan-Meier survival plot of nude mice bearing KB xenograft tumors treated with liposomal oxaliplatin 5a (POPC 65:30:5), oxaliplatin, or saline.

FIG. 10 shows the antitumor effects of liposomal oxaliplatin 5a compared to Eloxatin in mice bearing HT29 human colorectal xenografts. Mean tumor volume was measured after three weekly intravenous administrations of liposomal oxaliplatin 5a at 22 mg/kg/dose, free oxaliplatin at 15 mg/kg/dose (MTD) or saline (control). Values are mean±SEM for 5-10 mice/group.

7/11

FIG. 11 shows body weight changes of athymic nude mice bearing HT29 colorectal xenograft tumors after three weekly intravenous administrations of liposomal oxaliplatin 5a at 22 mg/kg/dose, free oxaliplatin at 15 mg/kg/dose (MTD), or saline (control). Values are mean±SEM for 5-10 mice/group.

FIG. 12 shows a Kaplan-Meier Plot showing percent survival of athymic nude mice bearing HT29 colorectal xenograft tumors treated with three weekly intravenous administrations of liposomal oxaliplatin 5a at 22 mg/kg/dose, free oxaliplatin at 15 mg/kg/dose (MTD) or saline (control). Liposomal oxaliplatin 5a increased survival significantly compared to Eloxatin and saline, p<0.05, Mantel-Cox, log-rank test. Each group started with 10 female mice bearing tumors.

8/11

FIG. 13 shows the antitumor effects of liposomal oxaliplatin 5a compared to Eloxatin in mice bearing HT29 human colorectal xenografts, Study II. Mean tumor volume was measured with three weekly intravenous administrations of liposomal oxaliplatin 5a at 15, 25, 35 mg/kg/dose, free oxaliplatin at 15 mg/kg/dose (MTD) or saline (control). Liposomal oxaliplatin 5a treatment significantly inhibited tumor growth compared to Eloxatin or saline treatment 30 days post initial dosing, p<0.05, one-way ANOVA, Newman-Keuls posthoc test. Values are mean±SEM for 5-10 mice/group.

FIG. 14 shows body weight changes of athymic nude mice bearing HT29 colorectal xenograft tumors with three weekly intravenous administrations of liposomal oxaliplatin 5a at 15, 25, 35 mg/kg/dose, free oxaliplatin at 15 mg/kg/dose (MTD) or saline (control). Values are mean±SEM for 5-10 mice/group.

9/11

FIG. 15 shows a Kaplan-Meier Plot showing percent survival of athymic nude mice bearing HT29 colorectal xenograft tumors treated with three weekly intravenous administrations of liposomal oxaliplatin 5a at 15, 25, 35 mg/kg/dose, free oxaliplatin at 15 mg/kg/dose (MTD) or saline (control). Each group started with 10 female mice bearing tumors.

FIG. 16 shows tumor platinum levels over time after dosing athymic nude mice with Eloxatin and liposomal oxaliplatin 5a. All doses are given as oxaliplatin molar equivalents. Data are represented as mean±standard error of three mice.

10/11

FIG. 17 shows plasma platinum levels over time after dosing athymic nude mice with Eloxatin and liposomal oxaliplatin 5a. All doses are given as oxaliplatin molar equivalents. Data are represented as mean±standard error of three mice.

FIG. 18 shows the antitumor effects of liposomal oxaliplatin 5a compared to Eloxatin in mice bearing BxPC-3 human pancreatic xenografts. Mean tumor volume was measured with three weekly intravenous administrations of liposomal oxaliplatin 5a at 15, 25, 35 mg/kg/dose, free oxaliplatin at 15 mg/kg/dose (MTD), or saline (control). Values are mean±SEM for 5-10 mice/group.

11/11

FIG. 19 shows body weight changes of athymic nude mice bearing BxPC-3 pancreatic xenograft tumors with three weekly intravenous administrations of liposomal oxaliplatin 5a at 15, 25, 35 mg/kg/dose, free oxaliplatin at 15 mg/kg/dose (MTD) or saline (control). Values are mean±SEM for 5-10 mice/group.

FIG. 20 shows a Kaplan-Meier Plot showing percent survival of athymic nude mice bearing BxPC-3 pancreatic xenograft tumors treated with three weekly intravenous administrations of liposomal oxaliplatin 5a at 15, 25, 35 mg/kg/dose, free oxaliplatin at 15 mg/kg/dose (MTD) or saline (control). Each group started with 10 female mice bearing tumors.

### DETAILED DESCRIPTION OF THE INVENTION

### I. General

The present invention relates to liposomal oxaliplatin compositions for cancer therapy. The liposome compositions described herein consist essentially of phosphatidylcholines, cholesterol, polyethylene glycol (PEG)-conjugated lipids, and encapsulated oxaliplatin. The disclosed compositions typically have a gel-to-fluid phase transition temperature lower than about 20° C. and demonstrate pH-dependent oxaliplatin release that is surprisingly rapid in acidic media. Methods for preparing the compositions and treatment of cancer with the compositions are also described. The compositions are particularly useful for enhancing intracellular oxaliplatin bioavailability in cancer cells and improving overall safety for cancer treatment. The compositions are broadly applicable for preventing and controlling cancers, providing a number of benefits to patients and clinicians.

### II. Definitions

As used herein, the term “liposome” encompasses any compartment enclosed by a lipid bilayer. The term liposome includes unilamellar vesicles which are comprised of a single lipid bilayer and generally have a diameter in the range of about 20 to about 400 nm. Liposomes can also be multilamellar, which generally have a diameter in the range of 1 to 10 μm. In some embodiments, liposomes can include multilamellar vesicles (MLVs; from about 1 μm to about 10 μm in size), large unilamellar vesicles (LUVs; from a few hundred nanometers to about 10 μm in size), and small unilamellar vesicles (SUVs; from about 20 nm to about 200 nm in size).

As used herein, the term “zwitterionic liposome” refers to liposomes containing lipids with both positively- and negatively-charged functional groups in the same lipid molecule. The overall surface charge of a zwitterionic liposome will vary depending on the pH of the external medium. In general, the overall surface charge of a zwitterionic liposome is neutral or negative at physiological pH (i.e., pH˜7.4).

As used herein, the terms “liposome size” and “average particle size” refer to the outer diameter of a liposome. Average particle size can be determined by a number of techniques including dynamic light scattering (DLS), quasi-elastic light scattering (QELS), and electron microscopy.

As used herein, the terms “molar percentage” and “mol %” refer to the number of a moles of a given lipid component of a liposome divided by the total number of moles of all lipid components. Unless explicitly stated, the amounts of active agents, diluents, or other components are not included when calculating the mol % for a lipid component of a liposome.

As used herein, the term “phosphatidylcholine lipid” refers to a diacylglyceride phospholipid having a choline headgroup (i.e., a 1,2-diacyl-sn-glycero-3-phosphocholine). The acyl groups in a phosphatidylcholine lipid are generally derived from fatty acids having from 6-24 carbon atoms. Phosphatidylcholine lipids can include synthetic and naturally-derived 1,2-diacyl-sn-glycero-3-phosphocholines.

As used herein, the term “cholesterol” refers to 2,15-dimethyl-14-(1,5-dimethylhexyl)tetracyclo[8.7.0.0^{2,7}.0^{11,15}]heptacos-7-en-5-ol (Chemical Abstracts Services Registry No. 57-88-5).

As used herein, the term “PEG-lipid” refers to a poly(ethylene glycol) polymer covalently bound to a hydrophobic or amphipilic lipid moiety. The lipid moiety can include fats, waxes, steroids, fat-soluble vitamins, monoglycerides, diglycerides, phospholipids, and sphingolipids. Preferred PEG-lipids include diacyl-phosphatidylethanolamine-N-[methoxy(polyethene glycol)]s and N-acyl-sphingosine-1-{succinyl[methoxy(polyethylene glycol)]}s. The molecular weight of the PEG in the PEG-lipid is generally from about 500 to about 5000 Daltons (Da; g/mol). The PEG in the PEG-lipid can have a linear or branched structure.

As used herein, the term “oxaliplatin” refers to [(1R,2R)-cyclohexane-1,2-diamine](ethanedioato-O,O′)platinum(II) (Chemical Abstracts Services Registry No. 63121-00-6).

As used herein, the term “composition” refers to a product comprising the specified ingredients in the specified amounts, as well as any product which results, directly or indirectly, from combination of the specified ingredients in the specified amounts. Pharmaceutical compositions of the present invention generally contain liposomal oxaliplatin as described herein and a pharmaceutically acceptable carrier, diluent, or excipient. By “pharmaceutically acceptable,” it is meant that the carrier, diluent, or excipient must be compatible with the other ingredients of the formulation and non-deleterious to the recipient thereof.

As used herein, the term “alkanol” refers to a C₁₋₄ alkane having at least one hydroxy group. Alkanols include, but are not limited to, methanol, ethanol, isoproponal, and t-butanol.

As used herein, the term “porous filter” refers to a polymeric or inorganic membrane containing pores with a defined diameter (e.g., 30-1000 nm). Porous filters can be made of polymers including, but not limited to, polycarbonates and polyesters, as well as inorganic substrates including, but not limited to, porous alumina.

As used herein, the term “sterile filtering” refers to sterilization of a composition by passage of the composition through a filter with the ability to exclude microorganisms and/or viruses from the filtrate. In general, the filters used for sterilization contain pores that are large enough to allow passage of liposomes through the filter into the filtrate, but small enough to block the passage of organisms such as bacteria or fungi.

As used herein, the term “cancer” refers to conditions including human cancers and carcinomas, sarcomas, adenocarcinomas, lymphomas, leukemias, and solid and lymphoid cancers. Examples of different types of cancer include, but are not limited to, lung cancer (e.g., non-small cell lung cancer or NSCLC), ovarian cancer, prostate cancer, colorectal cancer, liver cancer (i.e., hepatocarcinoma), renal cancer (i.e., renal cell carcinoma), bladder cancer, breast cancer, thyroid cancer, pleural cancer, pancreatic cancer, uterine cancer, cervical cancer, testicular cancer, anal cancer, pancreatic cancer, bile duct cancer, gastrointestinal carcinoid tumors, esophageal cancer, gall bladder cancer, appendix cancer, small intestine cancer, stomach (gastric) cancer, cancer of the central nervous system, skin cancer, choriocarcinoma, head and neck cancer, blood cancer, osteogenic sarcoma, fibrosarcoma, neuroblastoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, acute myelocytic leukemia, and multiple myeloma.

As used herein, the terms “treat”, “treating” and “treatment” refer to any indicia of success in the treatment or amelioration of a cancer or a symptom of cancer, including any objective or subjective parameter such as abatement; remission; diminishing of symptoms or making the cancer or cancer symptom more tolerable to the patient; or, in some situations, preventing the onset of the cancer. The treatment or amelioration of symptoms can be based on any objective or subjective parameter, including, e.g., the result of a physical examination or clinical test.

As used herein, the terms “administer,” “administered,” or “administering” refer to methods of administering the liposome compositions of the present invention. The liposome compositions of the present invention can be administered in a variety of ways, including parenterally, intravenously, intradermally, intramuscularly, or intraperitoneally. The liposome compositions can also be administered as part of a composition or formulation.

As used herein, the term “subject” refers to any mammal, in particular a human, at any stage of life.

As used herein, the term “about” indicates a close range around a numerical value when used to modify that specific value. If “X” were the value, for example, “about X” would indicate a value from 0.9X to 1.1X, and more preferably, a value from 0.95X to 1.05X. Any reference to “about X” specifically indicates at least the values X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.1X.

### III. Embodiments of the Invention

### Liposomes

In one aspect, the invention provides a composition for the treatment of cancer. The composition includes: (a) zwitterionic liposomes consisting essentially of from about 50 mol % to about 70 mol % of a phosphatidylcholine lipid or mixture of phosphatidylcholine lipids, from about 25 mol % to about 45 mol % of cholesterol, and from about 2 mol % to about 8 mol % of a PEG-lipid; and (b) oxaliplatin, encapsulated in the liposome in an amount such that the ratio of the total lipid weight to the oxaliplatin weight is from about 20:1 to about 65:1. In some embodiments, the phosphatidylcholine lipid or mixture of phosphatidylcholine lipids have fatty acid chains of 14 carbon atoms or more, and no more than one of the two fatty acid chains is unsaturated.

The liposomes of the present invention can contain any suitable phosphatidylcholine lipid (PC) or mixture of PCs. Suitable phosphatidylcholine lipids include saturated PCs and unsaturated PCs.

Examples of saturated PCs include 1,2-distearoyl-sn-glycero-3-phosphocholine (distearoylphosphatidylcholine; DSPC), 1,2-dipalmitoyl-sn-glycero-3-phosphocholine (dipalmitoylphosphatidylcholine; DPPC), 1-myristoyl-2-palmitoyl-sn-glycero-3-phosphocholine (MPPC), 1-palmitoyl-2-myristoyl-sn-glycero-3-phosphocholine (PMPC), 1-myristoyl-2-stearoyl-sn-glycero-3-phosphocholine (MSPC), 1-palmitoyl-2-stearoyl-sn-glycero-3-phosphocholine (PSPC), 1-stearoyl-2-palmitoyl-sn-glycero-3-phosphocholine (SPPC), and 1-stearoyl-2-myristoyl-sn-glycero-3-phosphocholine (SMPC).

Examples of unsaturated PCs include, but are not limited to, 1-palmitoyl-2-oleoyl-sn-glycero-3-phosphocholine (palmitoyloleoylphosphatidylcholine (POPC); 1-palmitoyl-2-linoleoyl-sn-glycero-3-phosphocholine, 1-stearoyl-2-oleoyl-sn-glycero-3-phosphocholine (SOPC), 1-stearoyl-2-linoleoyl-sn-glycero-3-phosphocholine, 1-oleoyl-2-myristoyl-sn-glycero-3-phosphocholine (OMPC), 1-oleoyl-2-palmitoyl-sn-glycero-3-phosphocholine (OPPC), and 1-oleoyl-2-stearoyl-sn-glycero-3-phosphocholine (DSPC).

Lipid extracts, such as egg PC, heart extract, brain extract, liver extract, soy PC, and hydrogenated soy PC(HSPC) are also useful in the present invention. In some embodiments, the phosphatidyl choline lipid or mixture of phosphatidylcholine lipids in the liposomes is other than hydrogenated soy phosphatidylcholine (HSPC) or other than a mixture comprising HSPC.

In some embodiments, the phosphatidylcholine lipid is selected from POPC, DSPC, SOPC, and DPPC. In some embodiments, the phosphatidylcholine lipid is POPC.

In general, the compositions of the present invention include liposomes containing 50-70 mol % of a phosphatidylcholine lipid or mixture of phosphatidylcholine lipids. The liposomes can contain, for example, 50, 51, 52, 53, 54, 55, 56, 57, 58, 59, 60, 61, 62, 63, 64, 65, 66, 67, 68, 69, or 70 mol % phosphatidylcholine. In some embodiments, the liposomes contain 50-55 mol % phosphatidylcholine. In some embodiments, the liposomes contain 55-70 mol % phosphatidylcholine. In some embodiments, the liposomes contain 65 mol % phosphatidylcholine. In some embodiments, the liposomes contain 60 mol % phosphatidylcholine. In some embodiments, the liposomes contain 55 mol % phosphatidylcholine.

The liposomes in the inventive compositions also contain 25-45 mol % of cholesterol (i.e., 2,15-dimethyl-14-(1,5-dimethylhexyl)tetracyclo[8.7.0.0^{2,7}.0^{11,15}]heptacos-7-en-5-ol). The liposomes can contain, for example, 25, 26, 27, 28, 29, 30, 31, 32, 33, 34, 35, 36, 37, 38, 39, 40, 41, 42, 43, 44, or 45 mol % cholesterol. In some embodiments, the liposomes contain 25-40 mol % cholesterol. In some embodiments, the liposomes contain 40-45 mol % cholesterol. In some embodiments, the liposomes contain 30 mol % cholesterol. In some embodiments, the liposomes contain 35 mol % cholesterol. In some embodiments, the liposomes contain 40 mol % cholesterol.

The liposomes of the present invention can include any suitable poly(ethylene glycol)-lipid derivative (PEG-lipid). In some embodiments, the PEG-lipid is a diacyl-phosphatidylethanolamine-N-[methoxy(polyethene glycol)]. The molecular weight of the poly(ethylene glycol) in the PEG-lipid is generally in the range of from about 500 Da to about 5000 Da. The poly(ethylene glycol) can have a molecular weight of, for example, 750 Da, 1000 Da, 2000 Da, or 5000 Da. In some embodiments, the PEG-lipid is selected from distearoyl-phosphatidylethanolamine-N-[methoxy(polyethene glycol)-2000] (DSPE-PEG-2000) and distearoyl-phosphatidylethanolamine-N-[methoxy(polyethene glycol)-5000] (DSPE-PEG-5000). In some embodiments, the PEG-lipid is DSPE-PEG-2000.

In general, the compositions of the present invention include liposomes containing 2-8 mol % of the PEG-lipid. The liposomes can contain, for example, 2, 3, 4, 5, 6, 7, or 8 mol % PEG-lipid. In some embodiments, the liposomes contain 4-6 mol % PEG-lipid. In some embodiments, the liposomes contain 5 mol % PEG-lipid.

In some embodiments, the zwitterionic liposome includes about 55 mol % POPC, about 40 mol % cholesterol, and about 5 mol % DSPE-PEG(2000). In some embodiments, the zwitterionic liposome includes about 60 mol % POPC, about 35 mol % cholesterol, and about 5 mol % DSPE-PEG(2000). In some embodiments, the zwitterionic liposome includes about 65 mol % POPC, about 30 mol % cholesterol, and about 5 mol % DSPE-PEG(2000).

In general, the compositions of the present invention contain liposome-encapsulated oxaliplatin in an amount such that a therapeutically effective dose of oxaliplatin can be delivered to a subject in a convenient dosage volume. The oxaliplatin content of a given formulation can be expressed as an absolution concentration (e.g., mg/mL) or as a relative amount with respect to the lipids in the liposomes. In general, the ratio of the total lipid weight to the oxaplatin weight is from about 20:1 to about 65:1. The lipid:oxaliplatin ratio can be, for example, 20:1, 25:1, 30:1, 35:1, 40:1, 45:1, 50:1, 55:1, 60:1, or 65:1. In some embodiments, oxaliplatin is encapsulated in said liposome in an amount such that the ratio of the total lipid weight to the oxaliplatin weight is from about 30:1 to about 45:1. In some embodiments, the composition of the invention includes liposomes containing oxaliplatin encapsulated in the liposomes in an amount such that the ratio of the total lipid weight to the oxaplatin weight is about 50:1. In some embodiments, the composition of the invention includes liposomes containing oxaliplatin encapsulated in the liposomes in an amount such that the ratio of the total lipid weight to the oxaplatin weight is from about 30:1 to about 35:1.

Liposome size can be determined by a number of methods known to those of skill in the art. Liposome size can be determined, for example, by dynamic light scattering (DLS), quasi-elastic light scattering (QELS), analytical ultracentrifugation, or electron microscopy. Liposome size can be reported in terms of liposome diameter, liposome volume, light-scattering intensity, or other characteristics. In some embodiments, the average particle size of a liposome corresponds to the volume mean value of the liposome. In some embodiments, the compositions of the present invention include zwitterionic liposomes having an average particle size of from about 75 to about 125 nm (diameter). For example, the liposomes can have a diameter of 75, 85, 90, 95, 100, 105, 110, 115, 120, or 125 nm. In some embodiments, the liposomes have an average particle size of 80-120 nm. In some embodiments, the liposomes have an average particle size of 90-120 nm. In some embodiments, the compositions of the invention contain liposomes have an average particle size of 90 nm.

### Methods for Preparing Liposomal Oxaliplatin

Liposomes can be prepared and loaded with oxaliplatin using a number of techniques that are known to those of skill in the art. Lipid vesicles can be prepared, for example, by hydrating a dried lipid film (prepared via evaporation of a mixture of the lipid and an organic solvent in a suitable vessel) with water or an aqueous buffer. Hydration of lipid films typically results in a suspension of multilamellar vesicles (MLVs). Alternatively, MLVs can be formed by diluting a solution of a lipid in a suitable solvent, such as a C₁₋₄ alkanol, with water or an aqueous buffer. Unilamellar vesicles can be formed from MLVs via sonication or extrusion through membranes with defined pore sizes. Encapsulation of oxaliplatin can be conducted by including the drug in the aqueous solution used for film hydration or lipid dilution during MLV formation.

Accordingly, some embodiments of the invention provide a composition containing zwitterionic liposomes as described above, wherein the liposomes are prepared by a method including: a) forming a lipid solution containing the phosphatidylcholine lipid, the cholesterol, the PEG-lipid, and a solvent selected from a C₁₋₄alkanol and a C₁₋₄alkanol/water mixture; b) mixing the lipid solution with an aqueous buffer to form multilamellar vesicles (MLVs); and c) extruding the MLVs through a porous filter to form small unilamellar vesicles (SUVs). In some embodiments, encapsulation of the oxaliplatin is conducted by including the oxaliplatin in the aqueous buffer during formation of the MLVs. Alternatively, encapsulation of the oxaliplatin can be conducted after extrusion to form the SUVs when there is low to substantially zero amount of cholesterol. In some embodiments, liposome preparation further includes sterile filtering the zwitterionic liposomes.

### Formulation and Administration

In some embodiments, the compositions of the invention can include a liposome as described above and a physiologically (i.e., pharmaceutically) acceptable carrier. The term “carrier” refers to a typically inert substance used as a diluent or vehicle for the liposomal oxaliplatin. The term also encompasses a typically inert substance that imparts cohesive qualities to the composition. Typically, the physiologically acceptable carriers are present in liquid form. Examples of liquid carriers include physiological saline, phosphate buffer, normal buffered saline (135-150 mM NaCl), water, buffered water, 0.4% saline, 0.3% glycine, glycoproteins to provide enhanced stability (e.g., albumin, lipoprotein, globulin, etc.), and the like. In some embodiments, the carrier includes carbohydrates such as, but not limited to, sucrose, dextrose, lactose, amylose, or starch. Since physiologically acceptable carriers are determined in part by the particular composition being administered as well as by the particular method used to administer the composition, there are a wide variety of suitable formulations of pharmaceutical compositions of the present invention (See, e.g., Remington's Pharmaceutical Sciences, 17^{th} ed., 1989).

The compositions of the present invention may be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, and calcium chloride. Sugars can also be included for stabilizing the compositions, such as a stabilizer for lyophilized liposome compositions.

Formulations suitable for parenteral administration, such as, for example, by intraarticular, intravenous, intramuscular, intratumoral, intradermal, intraperitoneal, and subcutaneous routes, include aqueous and non-aqueous, isotonic sterile injection solutions. The injection solutions can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, such as lyophilized liposomes. In the practice of the present invention, compositions can be administered, for example, by intravenous infusion, intraperitoneally, intravesically, or intrathecally. Parenteral administration and intravenous administration are preferred methods of administration. The formulations of liposome compositions can be presented in unit-dose or multi-dose sealed containers, such as ampoules and vials.

The pharmaceutical preparation is preferably in unit dosage form. In such form the preparation is subdivided into unit doses containing appropriate quantities of the active component, e.g., a liposome composition. The unit dosage form can be a packaged preparation, the package containing discrete quantities of preparation. The composition can, if desired, also contain other compatible therapeutic agents.

### Methods of Treating Cancer

In another aspect, the invention provides a method of treating cancer. The method includes administering to a subject in need thereof a composition containing liposomal oxaliplatin as described above. In some embodiments, the method includes administering a composition containing: (a) zwitterionic liposomes consisting essentially of from about 50 mol % to about 70 mol % of a phosphatidylcholine lipid or mixture of phosphatidylcholine lipids, from about 25 mol % to about 45 mol % of cholesterol, and from about 2 mol % to about 8 mol % of a PEG-lipid; and (b) oxaliplatin, encapsulated in the liposome in an amount such that the ratio of the total lipid weight to the oxaplatin weight is from about 20:1 to about 65:1. In some embodiments, the method includes administering a composition containing: a) zwitterionic liposomes consisting essentially of 55 mol % POPC, 40 mol % cholesterol, and 5 mol % DSPE-PEG(2000); and b) oxaliplatin, encapsulated in the liposome in an amount such that the ratio of the total lipid weight to the oxaplatin weight is about 50:1. In some embodiments, the method includes administering a composition containing: a) zwitterionic liposomes consisting essentially of 65 mol % POPC, 30 mol % cholesterol, and 5 mol % DSPE-PEG(2000); and b) oxaliplatin, encapsulated in the liposome in an amount such that the ratio of the total lipid weight to the oxaplatin weight is about 30:1 to about 40:1.

In therapeutic use for the treatment of cancer, the liposome compositions of the present invention can be administered such that the initial dosage of oxaliplatin ranges from about 0.001 mg/kg to about 1000 mg/kg daily. A daily dose range of about 0.01-500 mg/kg, or about 0.1-200 mg/kg, or about 1-100 mg/kg, or about 10-50 mg/kg, or about 10 mg/kg, or about 5 mg/kg, or about 2 mg/kg, or about 1 mg/kg can be used.

The dosages may be varied depending upon the requirements of the patient, the severity and type of the cancer being treated, and the liposome composition being employed. For example, dosages can be empirically determined considering the type and stage of cancer diagnosed in a particular patient. The dose administered to a patient should be sufficient to affect a beneficial therapeutic response in the patient over time. The size of the dose will also be determined by the existence, nature, and extent of any adverse side-effects that accompany the administration of a particular liposome composition in a particular patient. Determination of the proper dosage for a particular situation is within the skill of the practitioner. Generally, treatment is initiated with smaller dosages which are less than the optimum dose of the liposome composition. Thereafter, the dosage is increased by small increments until the optimum effect under circumstances is reached. For convenience, the total daily dosage may be divided and administered in portions during the day, if desired.

The methods described herein apply especially to solid tumor cancers (solid tumors), which are cancers of organs and tissue (as opposed to hematological malignancies), and ideally epithelial cancers. Examples of solid tumor cancers include bladder cancer, breast cancer, cervical cancer, colorectal cancer (CRC), esophageal cancer, gastric cancer, head and neck cancer, hepatocellular cancer, lung cancer, melanoma, neuroendocrine cancer, ovarian cancer, pancreatic cancer, prostate cancer and renal cancer. In one group of embodiments, the solid tumor cancer suitable for treatment according to the methods of the invention are selected from CRC, breast and prostate cancer. In another group of embodiments, the methods of the invention apply to treatment of hematological malignancies, including for example multiple myeloma, T-cell lymphoma, B-cell lymphoma, Hodgkins disease, non-Hodgkins lymphoma, acute myeloid leukemia, and chronic myelogenous leukemia.

The compositions used in the above methods may be administered alone, or in combination with other therapeutic agents. The additional agents can be anticancer agents or cytotoxic agents including, but not limited to, avastin, doxorubicin, cisplatin, oxaliplatin (in a non-liposome form), carboplatin, 5-fluorouracil, gemcitibine or taxanes, such as paclitaxel and docetaxel. Additional anti-cancer agents can include, but are not limited to, 20-epi-1,25 dihydroxyvitamin D3,4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caracemide, carbetimer, carboplatin, carboxamide-amino-triazole, carboxyamidotriazole, carest M3, carmustine, cam 700, cartilage derived inhibitor, carubicin hydrochloride, carzelesin, casein kinase inhibitors, castanospermine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethylnorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocarmycin SA, ebselen, ecomustine, edatrexate, edelfosine, edrecolomab, eflomithine, eflomithine hydrochloride, elemene, elsamitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, fluorocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ilmofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatin, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide 7, lobaplatin, lombricine, lometrexol, lometrexol sodium, lomustine, lonidamine, losoxantrone, losoxantrone hydrochloride, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, 06-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RII retinamide, RNAi, rogletimide, rohitukine, romurtide, roquinimex, rubiginone B1, ruboxyl, safingol, safingol hydrochloride, saintopin, sarcnu, sarcophytol A, sargramostim, SDI 1 mimetics, semustine, senescence derived inhibitor 1, sense oligonucleotides, signal transduction inhibitors, signal transduction modulators, simtrazene, single chain antigen binding protein, sizofuran, sobuzoxane, sodium borocaptate, sodium phenylacetate, solverol, somatomedin binding protein, sonermin, sparfosate sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer, or zorubicin hydrochloride. In some embodiments, the method can include administration of a drug selected from fluorouracil, leucovorin, and mixtures thereof.

### IV. Examples

### Example 1

### Preparation of Liposomal Platin Compositions

Encapsulation of oxaliplatin in liposomes was conducted via a solvent dilution procedure. Lipid mixtures were weighed in 100-mL glass bottles and dissolved in solutions of t-butanol (t-BuOH), ethanol (EtOH), and water, and heated at 70° C. until clear. Solutions generally contained 1:1 t-BuOH:EtOH (v:v) or 49:49:2 t-BuOH:EtOH:water (v:v:v), but the water content was adjusted depending on the specific amount of lipids used. Oxaliplatin was dissolved in pre-heated sucrose/acetate buffer (10 mM Sodium acetate, 300 mM Sucrose, pH 5.5; sterile filtered) at 70° C. Sonication was used when required. The lipid solution was added to the oxaliplatin solution with rapid mixing to form multi-lamellar vesicles (MLVs). An example preparation is summarized in Table 1.

The MLVs were passed through polycarbonate filters using a LIPEX™ Extruder (Northern Lipid Inc.) heated to 70° C. Extrusion was generally conducted using 3×80 nm stacked polycarbonate filters and a drain disc in an 800 mL extruder. The number of filters was adjusted as necessary, depending on the lipid composition being extruded. Following each pass through the extruder, vesicle sizes and size distributions were determined using a quasi-elastic light scattering (QELS) particle size analyzer. The extrusion was stopped after a mean volume diameter of 90-120 nm was achieved. Following extrusion, the liposomes were diluted 10-fold with cold (2-15° C.) sucrose/acetate buffer. 400 mL of liposomes were diluted with 3600 mL cold buffer. Dilution can prevent precipitation of any unencapsulated oxaliplatin during subsequent processing. The liposomes were then concentrated via ultrafiltration to a concentration of roughly 50 mg/mL lipid.

TABLE 1
Preparation of Oxaliplatin Multi-lamellar Vesicles

| Lipid Solution | |
|---|---|
| Total lipid (g) | 20.00 |
| t-BuOH:EtOH:H₂O (49:49:2 v/v/v) | 40.0 |
| (mL) | |
| Total lipid/solvent (mg/mL) | 500 |
| Oxaliplatin Solution | |
| Oxaliplatin (g) | 4.32 |
| 300 mM Sucrose/Acetate buffer | 360.0 |
| (mL) | |
| Oxaliplatin solution (mg/mL) | 12.0 |
| MLV Solution | |
| Final volume (mL) | 400.0 |
| Final drug (mg/mL) | 10.8 |
| Final lipid (mg/mL) | 50 |
| Total solvent (v/v %) | 10% |

Diafiltration was conducted to exchange the external buffer and concentrate the liposomes, and to remove unencapsulated oxaliplatin and residual organic solvents. The diafiltration system included a Masterflex pump with an L/S pumphead and 36-gauge tubing. In general, a peristaltic pump capable of maintaining 10 psig at the inlet of the cartridge can be used. The diafiltration system also included 500-kDa cartridges, with roughly 55 cm² surface area per gram of lipid. For example, two Spectrum M4-500S-260-01N PS 615 cm² cartridges in series can provide adequate surface area for filtration of a preparation containing 20 grams of lipids. The system was rinsed thoroughly with at least 500 mL purified water and then with at least 200 mL of 1300 mM sucrose/acetate buffer. Volumes were adjusted based on the size of cartridges used. The concentrated liposomes (50 mg/mL) were diafiltered against 10 wash volumes of buffer (10 mM acetate, 300 mM Sucrose pH 5.5). Ultrafiltration was conducted again to achieve a lipid concentration of roughly 90 mg/mL. Portions of the preparations were reserved for particle sizing and analysis.

Sterile filtration of the compositions was conducted using 0.2-μm syringe filters equipped with cellulose acetate membranes (e.g., 0.20 μm MiniSart, surface area=6.2 cm²; 0.20 μm Sartorius Sartobran 150, surface area=150 cm²). Filters were replaced as necessary; in general, one square centimeter of membrane was found to adequately filter 3 to 10 mL of a composition. When necessary, dilution of the composition with sterile buffer was conducted to obtain a desired oxaliplatin concentration (e.g., 1 mg/mL) before sterile filtration. Sterile depyrogenated vials were filled with the compositions using a sterile pipette. The vials were capped with autoclaved butyl stoppers and crimped aluminum seals and stored at 2-8° C.

The method described above was used to prepare the compositions summarized in Table 2.

TABLE 2
Liposomal Oxaliplatin Compositions

| | Example | | | | |
|---|---|---|---|---|---|
| | 1a | 1b | 1c | 1d | 1e |
| Lipid Composition | DSPC:Chol:DSPE-PEG^{a} | DPPC:Chol:DSPE-PEG | DMPC:Chol:DSPE-PEG | POPC:Chol:DSPE-PEG | DSPC:Chol:DSPG |
| (molar ratio) | (55:40:5) | (55:40:5) | (55:40:5) | (55:40:5) | (70:10:20) |
| Total lipid | 76.8 | 84.6 | 86 | 82.5 | 82.40 |
| concentration | | | | | |
| (mg/mL) | | | | | |
| Oxaliplatin | 3.62 | 4.18 | 3.93 | 4.05 | 4.66 |
| concentration | | | | | |
| (mM) | | | | | |
| Oxaliplatin | 1.44 | 1.66 | 1.56 | 1.61 | 1.85 |
| concentration | | | | | |
| (mg/mL) | | | | | |
| Particle size, | 104.3 | 101.8 | 118.6 | 89.4 | 100.2 |
| volume | | | | | |
| (nm) | | | | | |
| Particle size, | 167, 60.4 | 163, 63.0 | 193, 67.1 | 132, 57.9 | 169, 54.9 |
| 90%, 10% | | | | | |
| (nm) | | | | | |
| Zeta potential | −1.24 | −1.39 | −0.91 | −0.81 | −23.3 |
| (mV) | | | | | |
| Lipid:oxaliplatin | 53 | 51 | 55 | 51 | 45 |
| (wt:wt) | | | | | |

^{a}PEG molecular weight = 2000 Da.

The method described above was also used to prepare oxaliplatin and cisplatin compositions as summarized in Table 3.

TABLE 3
Liposomal Platin Compositions

| | | | | | Platin |
|---|---|---|---|---|---|
| | | | Particle | Zeta | concen- |
| | Lipid Composition | | size | potential | tration |
| Example | (molar ratio) | Platin | (d · nm) | (mV) | (mg/mL) |
| 1f | DSPC: | Cisplatin | 87.41 | −0.879 | 1.65 |
| | Chol: | | | | |
| | DSPE-PEG^{a} | | | | |
| | (55:40:5) | | | | |
| 1g | POPC: | Cisplatin | 88.11 | −0.984 | 2.06 |
| | Chol: | | | | |
| | DSPE-PEG | | | | |
| | (60:35:5) | | | | |
| 1h | POPC: | Cisplatin | 86.47 | −0.638 | 2.03 |
| | Chol: | | | | |
| | DSPE-PEG | | | | |
| | (65:30:5) | | | | |
| 1i | POPC: | Oxaliplatin | 80.57 | −6.99 | 1.84 |
| | Chol: | | | | |
| | DSPE-PEG | | | | |
| | (60:35:5) | | | | |
| 1j | POPC: | Oxaliplatin | 81.71 | −7.4 | 1.91 |
| | Chol: | | | | |
| | DSPE-PEG | | | | |
| | (65:30:5) | | | | |

^{a}PEG molecular weight = 2000 Da.

### Example 2

### In Vitro Release of Platin Drug from Liposomes

The in vitro release of oxaliplatin from liposomes in Examples 1a-1e (Table 2) was studied at pH 7.1. As shown in 1/11

FIG. 1, the release profiles indicated that POPC-based formulation 1d had the highest release rate in comparison to other formulations using saturated lipids. No significant difference was observed for the other four oxaliplatin formulations.

Examples 1f-1j, containing either cisplatin or oxaliplatin (Table 3), were compared with respect to platin release rate. In vitro release was determined at pH 5.0 and pH 7.1. As shown in Table 4, POPC-based formulations containing oxaliplatin (1i and 1j) exhibit pH-dependent release rates while other formulations containing cisplatin do not. Oxaliplatin formulations also exhibit faster and higher release than cisplatin formulations. Data in Table 4 are plotted in 2/11

### FIG. 2 and FIG. 3.

Taking the release data for POPC-based formulations together, the release rates of oxaliplatin at 48 hrs was about 10% for liposomes containing POPC:Chol:DSPE-PEG (55:40:5) at pH 7.1, but 20% and 30% for liposomes containing POPC:Chol:DSPE-PEG (60:35:5 and 65:30:5, respectively), at pH 5.0. As shown here, the POPC content and POPC/cholesterol ratio of the liposomes, as well as the pH-dependent characteristics of oxaliplatin, have been found to contribute to the enhanced release of oxaliplatin in acidic media.

TABLE 4
In vitro release (%) of platin drugs from liposome compositions over time.

| | Composition/pH | | | | | | | | | |
|---|---|---|---|---|---|---|---|---|---|---|
| | 1f/ | 1g/ | 1h/ | 1i/ | 1j/ | 1f/ | 1g/ | 1h/ | 1i/ | 1j/ |
| | 7.4 | 7.4 | 7.4 | 7.4 | 7.4 | 5.0 | 5.0 | 5.0 | 50 | 5.0 |
| 0 hrs. | 1.72 | 2.55 | 7.25 | 2.19 | 8.46 | 1.91 | 2.81 | 7.09 | 2.20 | 8.87 |
| 1 hrs. | 2.58 | 2.90 | 7.44 | 2.58 | 9.24 | 2.57 | 2.98 | 7.41 | 2.62 | 9.37 |
| 6 hrs. | 2.90 | 3.65 | 8.73 | 3.10 | 9.70 | 2.93 | 3.84 | 8.78 | 4.48 | 11.80 |
| 24 hrs. | 3.25 | 5.55 | 11.00 | 5.68 | 13.80 | 3.33 | 6.43 | 11.70 | 12.41 | 20.23 |
| 48 hrs. | 3.83 | 7.99 | 14.12 | 8.00 | 14.43 | 3.84 | 9.41 | 15.99 | 21.13 | 29.34 |

### Example 3

### Physical Characterization of Liposomal Compositions

The phase transition temperature (Tₘ) of for the gel-to-fluid phase transition was determined for liposomes with varying lipid content, as shown in Table 5. A distinct phase transition temperature was detected for mixtures containing 55-95% saturated phosphatidyl choline (DPPC, DSPC, or HSPC), 0-40 mol % cholesterol, and 5 mol % DSPE-PEG. Tₘ values were in the range of about 41-56° C., much higher than ambient temperature or physiological temperature. In contrast, there was no detectable transition peak for the POPC-based formulation. The gel-liquid crystalline thermal transition temperature of POPC is around −2° C. Transition temperatures for binary mixtures of POPC and cholesterol have been reported to be much below 0° C.

TABLE 5
Phase Transition Temperatures for Liposome Formulations

| Liposome Components | Mole Ratio | Tₘ (° C.) |
|---|---|---|
| DPPC/DSPE-PEG^{a} | 95/5 | 43.1 |
| DPPC/Cholesterol/DSPE-PEG | 80/10/5 | 41.8 |
| DSPC/DSPE-PEG | 95/5 | 54.9 |
| HSPC/Cholesterol/DSPE-PEG | 57/38/5 | 49.7 |
| DSPC/DPPC/Chol/DSPE-PEG | 46.25/18.75/30/5 | 53.4 |
| POPC/Cholesterol/DSPE-PEG | 55/40/5 | No Tₘ detectable |
| | | <20° C. |

^{a}PEG molecular weight = 2000 Da.

Overall, in view of the platin release data and the phase transition behavior of selected liposome compositions, the advantageous properties of the inventive compositions are believed to arise at least in part from a combination of membrane mechanics and pH-dependent charge state.

Liposome nanoparticles are particularly suitable for delivering therapeutic agents to solid tumor sites via the “enhanced permeability and retention” (EPR) effect (V. P. Torchilin. The AAPS Journal. 9 (2): Article 15. 2007). Solid tumors rely heavily on hyperactive angiogenesis in sustaining the high demands for oxygen and nutrients in the cancer cells. It is well known these tumors exhibit porous fenestrations within the membranous structures of their vasculature, providing an excellent pathway for nanoparticles in a certain size range to be delivered preferentially to the tumor sites. Liposome nanoparticles in the size range of about 50-150 nm are particularly suitable for taking advantage of this phenomenon for drug delivery.

The endosomal-lysosomal process is believed to be the major route responsible for internalization and intracellular digestion of nanoparticles like liposomes (Desnick, R. J. & Schuchman, E. H. Nature Reviews Genetics. 3: 954-966. 2002). As a result of the process, most extracellular nanoparticles are internalized by endocytosis to form early endosomes, which move from the plasma membrane towards the cell nucleus. As they do so, they become acidic and give rise to ‘late’ endosomes. This increasing acidity leads to the dissociation of lysosomal enzymes from mannose-6-phosphate receptors. Late endosomes also fuse with primary lysosomes (which contain lysosomal hydrolases and bud from the Golgi) to form secondary lysosomes. The distinction between late endosomes and lysosomes is based primarily on pH. The lysosome is a more acidic compartment, in which most macromolecular degradation occurs. The size of the liposomes in the compositions of the present invention, coupled with their surprisingly rapid release of oxaliplatin in acidic media, are particularly useful for capitalizing on the EPR effect and the endosomal-lysosomal internalization process for selectively delivering oxaliplatin to cancer tissues.

### Example 4

### Effects of Liposome Composition on Oxaliplatin Release Rate and Efficacy

### Methods

Preparation of Oxaliplatin Formulation E000201-001.

Into a 20 mL scintillation vial was added 581 mg POPC (1-hexadecanoyl-2-(9Z-octadecenoyl)-sn-glycero-3-phosphocholine, Lipoid, FW=760, 0.76 mmol), 407 mg of cholesterol (Fisher, FW=386.7, 1.05 mmol) and 263 mg of DSPE-PEG(2000) (1,2-distearoyl-sn-glycero-3-phosphoethanolamine-N-[methoxy(polyethylene glycol)-2000], Lipoid, FW=2749, 0.10 mmol). This was dissolved into 2.5 mL EtOH (lipids dissolved in EtOH at 65° C. and at ambient temperature is a paste).

Into a 60 mL amber bottle was added 400 mg oxaliplatin (LC labs, 99% FW=397, 1 mmol) and 25 mL of aqueous 0.3 M sucrose solution. The oxaliplatin solution was heated to 65° C. in a temperature controlled water bath. To the heated solution was added the EtOH solution of lipids giving a milky white suspension. Heating continued at 65° C. for 30 min in the water bath.

The vesicles above were extruded 5× through 0.1 micron double stacked membranes (Whatman, Nuclepore Track-Etched, extrusion carried out in isolator) at 65° C. using a 100 mL Lipex™ extruder under 200-600 psig nitrogen.

The resulting liposomes were chilled at 5° C. overnight which caused crystallization of excess oxaliplatin. The liposomes were filtered from the crystalline oxaliplatin using a 0.45 micron Nylon filter. The filtrate was diafiltered against 300 mL 0.3 M sucrose, containing 20 mM acetate buffer, (pH 6.1) using mPES 500 KDa MWCO hallow fibers (KrosFlo Research II model tangential flow diafiltration unit). The final volume of the retained liposomes was ca. 15 mL and was stored in amber glass serum vials (rubber stopper) at 5° C.

Particle size and zeta potential were determined (50 uL diluted to 1 mL with pH 7 PBS) using a Malvern zeta sizer (DLS) and reported as volume mean values in nm.

Lipids were analyzed via HPLC while Pt was quantified by ICP-MS. “Free” Pt was determined by ICP-MS of the filtrate obtained from 30 KDa Amicon centrifuge filters (9000 rpm for 10 min at ambient temperature).

Preparation of Oxaliplatin Formulations E000201-002 Thru 005 and E000201-008 and 009.

Procedures for the preparation of liposomes E000201-002 thru 005 and E000201-008 and 009 were identical to those used to prepare E000201-001 with different amounts of lipids to obtain variable ratios of POPC to Cholesterol while maintaining a 5% (molar) amount of DSPE-PEG(2000).

Procedure for Analysis of Oxaliplatin Liposome Formulations: POPC, Cholesterol, DSPE-PEG(2000).

Concentrations (in μg/mL) were determined for Cholesterol, POPC, DSPE-PEG(2000) and Lyso-DSPC in liposomal drug product formulations using a reverse phase HPLC method using a Waters Xselect reverse phase column with an ELSD detector. The column was an X Select CSH C18, 3.5 μm, 3.0×150 mm (PN: 186005263). Column temperature was 50° C. and the autosampler temperature was 10° C. Injections of 10 μL were made and separated using a 25-min chromatography program at a flow rate of 1.0 mL/min. The voltage range in Totalchrom was 2 volts. The Alltech ELSD was operated with the drift tube at 80° C., using a gas pressure of 1.5 L/min with a gain set to 4. Mobile Phase A (25 mM ammonium formate in H₂O) and Mobile Phase B (20% acetonitrile in methanol) were used in the gradient program outlined in Table 6.

TABLE 6
Gradient HPLC conditions

| Time | | |
|---|---|---|
| (min) | % A | % B |
| initial | 15 | 85 |
| 12.00 | 10 | 90 |
| 15.00 | 0 | 100 |
| 20.00 | 0 | 100 |
| 20.01 | 15 | 85 |
| 25.00 | 15 | 85 |

20 μL aliquots of formulations were weighed into a tared microfuge vial and 80 μL of n-propanol were added. The vials were vortexed and sonicated for 20 minutes. Dilutions at 1:50 and 1:100 were prepared using n-propanol as the diluent. Lipid Stock Standards and Working Standards were prepared in n-propanol. Lipid standard curves (6 samples per series) were prepared by serial dilution. Upper-limit (51) concentrations were: 900 μg/mL for phosphatidylcholines and phosphatidylglycerols; 700 μg/mL for cholesterol; 400 μg/mL for DSPE-PEG(2000); and 150 μg/mL for lysophospholipids. Dilutions were performed according to Table 7.

TABLE 7
Preparation of Lipid Standards.

| | Vol of S1, | Vol of n-propanol, |
|---|---|---|
| Standard | μL | μL |
| S1 | 500 | None |
| S2 | 400 | 100 |
| S3 | 300 | 200 |
| S4 | 200 | 300 |
| S5 | 100 | 400 |
| S6 | 50 | 450 |

Release of Oxaliplatin from Liposomes.

Release rates of Oxaliplatin from the liposomal drug product were determined by membrane dialysis followed by ICP-MS analysis. This method separates free (released) Oxaliplatin from encapsulated Oxaliplatin using a dialysis membrane. The receiver fluid is then analyzed by ICP-MS to determine the Platinum concentration which is converted to Oxaliplatin equivalents. Three in vitro release assays address different aspects of stability of the liposomal formulations. Physiological release was measured using PBS pH 7 at 37° C. to mimic in vivo conditions. By lowering the pH of PBS to 5, the release reflected endosomal conditions within the cell. Biological release using FBS provided release data in the presence of relevant protein concentrations.

A Float-A-Lyzer membrane was preconditioned by adding 0.5 mL PBS pH 7 into the membrane. The membrane was allowed to pre-condition for at least 10 min prior to addition of formulations. The membrane was inverted periodically to ensure that the entire membrane area was pre-conditioned. A thermoshaker was preheated to 37° C. and the shaking speed was set to 400 rpm.

0.5 mL of liposomal formulations were loaded into Float-A-Lyzer membranes. 15-mL portions of release solution (PBS pH 7, PBS pH 5, or FBS) were added to 50-mL conical tubes. Float-A-Lyzers were inserted into conical tubes, and the assemblies were placed into the thermoshaker. Samples were collected periodically using the sample collection schedule summarized in Table 8. 100 μL of sample at each timepoint was transferred to a pre-labeled deep well plate. The deep well plates were sealed and stored in a refrigerator between collection time points.

TABLE 8
Sample Collection Schedule.

| | | | | |
|---|---|---|---|---|
| Time (hrs) | 6 | 24 | 30 | 48 |
| Volume (μL) | 100 | 100 | 100 | 100 |

Determination of Total Platinum in Samples by ICP-MS.

The concentration of platinum (Pt) was measured by ICP-MS (inductively coupled plasma mass spectrometry) using a PerkinElmer Inductively Coupled Plasma Mass Spectrometer (NexION300q ICP-MS) equipped with a sample introduction system (including a Meinhard concentric nebulizer, low volume quartz cyclonic spray chamber and quartz torch), an RF generator excitation source, a mass spectrometer with gold metalized ceramic quadrupoles and SimulScan Dual stage Detector (electron multiplier), and an S10 Autosampler

Calibration Working Standard Preparation.

Platinum working standards (1000 ng/mL and 10 ng/mL) were prepared by serial dilution with 1% nitric acid from a 1000 μg/mL standard solution. An iridium internal standard stock solution (200 ng/mL) was prepared in 1% nitric acid. Calibration working standard solutions were prepared by diluting the 10 ng/mL Pt & 1000 ng/mL Pt stock standard solutions and the 200 ng/mL Ir internal standard solutions. Standards were prepared as outlined in Table 9.

TABLE 9
Preparation of ICP standards.

| | Pt | Ir |
|---|---|---|
| | pg/mL | ng/mL |
| Blank | 0 | 2 |
| Std 1 | 10 | 2 |
| Std 2 | 50 | 2 |
| Std 3 | 100 | 2 |
| Std 4 | 500 | 2 |
| Std 5 | 1000 | 2 |
| Std 6 | 5000 | 2 |
| Std 7 | 10000 | 2 |

Sample Preparation and Analysis.

10 μL of sample was diluted with 5 mL nitric acid, and the samples were heated at 70° C. overnight to ensure complete digestion. The samples were then diluted with 45 mL of water resulting in a 500× dilution. A 200,000× dilution was prepared from the 500× dilution using 10% nitric acid, and the iridium internal standard was added at the desired concentration. ICP-MS was conducted using the operating parameters outlined in Table 10. Percent release of oxaliplatin was calculated as: Conc Oxal (t=6 hr)/Conc Oxal (final)

TABLE 10
ICP-MS Operating Parameters

| Method Parameter | Setting |
|---|---|
| Conditions | |
| RF Power: | 1000 W |
| Plasma Flow: | 17.0 L/min |
| Auxiliary Flow: | 1.20 L/min |
| Nebulizer Flow: | 0.89 L/min |
| Timing | |
| Sweeps/Reading: | 20 s |
| Readings/Replicate: | 3 |
| Replicates: | 3 |
| Scan Mode: | Peak Hopping |
| Dwell Time (per amu): | 50 ms |
| Processing: | Dual Detector, AutoLens ON |
| Sampling | |
| Sample Flush: | 60 s Speed −48 rpm |
| Read Delay: | 30 s Speed −20 rpm |
| Wash: | 60 s Speed −48 rpm |
| Analyte: | Pt 194.965 amu |
| Internal Standard: | Ir 190.961 amu |
| Curve Type: | Linear, Thru Zero |

Determination of In Vitro IC₅₀ of Liposomal Oxaliplatin in HT29 Cells.

HT-29 human colorectal adenocarcinoma cells (#HTB-38, ATCC, Manassas, Va.) were plated in 96-well tissue culture plates (Costar #3595) at 5×10³ cells/well in a final volume of 0.1 mL of 10% fetal bovine serum in McCoy's 5A (#10-050-CV, Mediatech, Manassas, Va.). Defined fetal bovine serum was obtained from HyClone (#SH30070.03, lot #AWB96395, Logan, Utah). Plates containing cells were incubated at 37° C. in 5% CO₂ in humidified air for 24 hr. The selected initial cell plating density was chosen based upon the approximate doubling time of the human tumor cell line.

Test compounds were diluted from stock solutions to 2.2 mmol/L in Dulbecco's modified phosphate-buffered saline (DPBS; Mediatech, Inc., lot #21031339, Manassas, Va.), then serially diluted three-fold in DPBS to generate a nine point dose-response curve. Ten microliters of diluted test compounds were added to wells in triplicate to achieve the desired final concentration of test compounds. Plates containing cells with and without added test compounds were returned to incubation as described above.

For the two hour cytotoxicity assessment, medium was removed after two hours of drug exposure and replaced with 0.1 mL/well culture medium, and cells were incubated for an additional 70 hours as above.

For the twenty-four hour cytotoxicity assessment, medium was removed after one day of drug exposure and replaced with 0.1 mL/well culture medium, and cells were returned to incubation for an additional 48 hours. Subsequently, cell viability was assessed using Alamar Blue. For this purpose, media was removed by pipetting from cultured cells and replaced with 0.1 mL/well of 10% (v/v) Alamar Blue (#BUF012A, AbD Serotec, Raleigh, N.C.) diluted in the appropriate cell culture media. Plates were then returned to incubation as before for appropriate color development, between two to four hours.

Fluorescence of individual plate wells was measured at 545 nm/590 nm (excitation/emission) using a BioTek Synergy4 microplate reader. Cell viability was calculated as a percentage of measured fluorescence obtained relative to cells treated with culture media alone. IC₅₀ values (umol/L) were determined with the mean of triplicate values using nonlinear regression analysis and a four-parameter logistic model

### Results

The results obtained for samples comprising 0 to 56% Cholesterol, 5% DSPE-PEG(2000), and a balance of POPC is summarized in Table 11.

TABLE 11
Physical Properties of Liposomes with Component Assay Values

| | particle | zeta | | | | | | | |
|---|---|---|---|---|---|---|---|---|---|
| | size | potential | | | | | total | | % |
| | (Volume | pH 7.0 | final | mol % | Chol | mol % | mg/mL | Pt | Free |
| Sample # | mean nm) | (mV) | pH | PC | mol % | PEG | lipids | ug/mL | Pt |
| 4a | 100.2 | −1.19 | 6.09 | 39.0 | 56.3 | 4.7 | 50.6 | 1513 | 2% |
| 4b | 92.2 | −1.42 | 6.05 | 50.2 | 44.6 | 5.2 | 41.7 | 1144 | 3% |
| 4c | 80 | −1.29 | 6.08 | 74.6 | 20.7 | 4.7 | 57.2 | 1474 | 15% |
| 4d | 78.9 | −1.11 | 6.08 | 84.0 | 10.7 | 5.3 | 48.6 | 1034 | 13% |
| 4e | 74.8 | −0.88 | 6.05 | 95.0 | 0.0 | 5.0 | 58.3 | 1408 | 19% |
| 4f | 80.7 | −0.59 | 6.11 | 65.3 | 29.6 | 5.1 | 52.7 | 1469 | 4% |
| 4g | 84.8 | −1.38 | 6.11 | 54.5 | 40.6 | 5.0 | 63.7 | 1685 | 2% |

The release of Oxaliplatin from liposomes was determined using three in vitro release assays which address different aspects of stability of the liposomal formulations. Physiological release is measured using PBS pH 7 at 37° C. to mimic in vivo conditions. By lowering the pH of PBS to 5, the release reflects endosomal conditions observed within the cell. Biological release using FBS provides release data in the presence of relevant protein concentrations.

The results are given in Table 12 for the three different media examined at the final time point (48 hr). An example of the time release behavior is shown in FIG. 4 for liposomal oxaliplatin 4a.

TABLE 12
Release of Oxaliplatin and IC₅₀ values for
POPC:Cholesterol:DSPE-PEG(2000) Liposomes

| | Release | Release | Release | HT29 | HT29 |
|---|---|---|---|---|---|
| Sam- | 37 C. pH | 37 C. | 37 C. | 2 hr cell | 24 hr cell |
| ple | 7.4 PBS | pH 5 PBS | FBS | kill IC50 | kill IC50 |
| # | (48 hr) | (48 hr) | (48 hr) | [μM] | [μM] |
| 4a | 2.0% | 3.0% | 4.0% | 84.9 | 30.25 |
| 4b | 4.0% | 7.9% | 7.0% | 204.8 | 20.12 |
| 4c | 19.0% | 28.0% | 17.0% | 60.1 | 5.84 |
| 4d | 18.0% | 23.0% | 39.0% | 53.7 | 3.18 |
| 4e | 27.0% | 32.0% | 28.0% | 36.6 | 2.65 |
| 4f | 10.0% | 10.0% | 15.0% | 83.1 | 8.70 |
| 4g | 3.0% | 5.0% | 9.0% | 96.5 | 18.90 |

Correlation of In Vitro Results to Composition of Liposome.

The release of oxaliplatin from liposomes consisting of POPC, cholesterol and DSPE-PEG(2000) as a function of the molar % cholesterol in the formulation is shown graphically in 4/11

FIG. 5 for data obtained in PBS, pH 7.4 after 48 hrs at 37° C.

The IC₅₀ values obtained from liposomal oxaliplatin consisting of POPC, cholesterol and DSPE-PEG(2000) as a function of the molar % cholesterol in the formulation is shown graphically in FIG. 6 for HT29 cells after 24 hrs exposure.

Efficacy testing has been carried out using the liposomal oxaliplatin formulation containing POPC, cholesterol and DSPE-PEG(2000) in 65:30:5 molar ratios. Results from in vivo studies (as determined by tumor size growth delay and survival) indicated greater efficacy than that obtained with Eloxatin (current commercial product of Oxaliplatin). The importance of the molar ratio of 65:30:5 was investigated by variation in the POPC:cholesterol ratio. As potential surrogates for efficacy, the in vitro tests for oxaliplatin release and cell proliferation inhibition were carried out on seven different ratios of POPC:cholesterol.

As evidenced by the high correlations obtained, both the release rate of oxaliplatin from the liposome and the IC₅₀ against HT29 cells were dependent on the molar ratio of POPC to cholesterol. The release of oxaliplatin from the liposome increases as the ratio increases (higher POPC, lower cholesterol). The IC₅₀ potency is enhanced upon increasing the ratio (higher POPC, lower cholesterol). As such, the IC₅₀ decreases with a higher release rate of oxaliplatin as shown in 5/11

### FIG. 7.

Without wishing to be bound by any particular theory, it is believed that release of a greater amount of oxaliplatin over the span of 48 hrs can lead to higher toxicity with lower efficacy due to less accumulation of drug at the tumor (drug lost to circulation and elimination). A lower release of oxaliplatin over 48 hrs can result in lower efficacy due to too low a concentration of bio-available oxaliplatin (oxaliplatin encapsulated in the liposome is thought to be non-biologically active).

### Example 5

### In Vivo Study of Liposomal Oxaliplatin Efficacy

### Single Agent Efficacy Studies

A number of commercially available and privately-acquired tumor cell lines were initially surveyed for their sensitivity to various platinum agents, including oxaliplatin. Among a panel of commercially-available human colon tumor cell lines tested, HCT-116 cells (0.4 uM IC₅₀, 72 h) were identified, which have enhanced oxaliplatin in vitro cytotoxicity compared to HT29 and several other cell lines (˜5-10 uM IC₅₀, 72 h). The cell lines were also surveyed for sensitivity to 5FU. IC₅₀ values of ˜7-10 uM @72 h were obtained for all cell lines tested except HT29 (>50 uM, IC₅₀, 72 h). In vitro synergy was evaluated with oxaliplatin and 5FU for all the surveyed cell lines, however, no cell lines were identified where addition of the paired agents resulted in a markedly improved cytotoxicity. Additionally, literature survey was conducted in search of in vitro-generated oxaliplatin-resistant colorectal cell lines to potentially include pre-clinical pharmacology studies in an effort to show improved sensitivity of these cells to a liposomal vs. free oxaliplatin treatment. From this survey, three other oxaliplatin-resistant colorectal tumor lines were identified; HT29 (Plascencia et al., 2006; Yang et al., 2006), and DLD-1 (Kashiwagi et al., 2011).

Based upon the preliminary in vitro studies and the literature surveyed, initial testing of the novel liposomal oxaliplatin formulation liposomal oxaliplatin 5a in a series of xenograft models was conducted. Liposomal oxaliplatin 5a includes POPC, cholesterol and DSPE-PEG(2000) in 65:30:5 molar ratios. These studies included single and multi-dose regimens and multiple dosage levels.

TABLE 13
Liposomal oxaliplatin 5a single agent efficacy and biodistribution
studies in human xenograft models

| | Efficacy Studies | Dosing |
|---|---|---|
| 1 | Antitumor effects of liposomal oxaliplatin | Single Dose |
| | 5a in mice bearing KB xenografts | (40 & 60 mg/kg) |
| 2 | Antitumor effects of liposomal oxaliplatin | 3 weekly doses |
| | 5a in mice bearing HT29 human colorectal | (15, 25, 35 mg/kg) |
| | xenografts | |
| 3 | Biodistribution of liposomal oxaliplatin 5a | |
| | in mice bearing HT29 human colorectal | |
| | xenografts | |
| 4 | Antitumor effects of liposomal oxaliplatin | 3 weekly doses |
| | 5a in mice bearing BxPC-3 human | (15, 25, 35 mg/kg) |
| | pancreatic xenografts | |
| 5 | Antitumor effects of liposomal oxaliplatin | 3 weekly doses |
| | 5a in mice bearing HCT-116 or Colo205 | (15, 25, 35 mg/kg) |
| | human colorectal xenografts | |

KB (epidermoid oral carcinoma human tumor) cells have been reported to retain their sensitivity to oxaliplatin while exhibiting inherent resistance to cisplatin. Among cisplatin-resistant cell lines, IC₅₀ values for oxaliplatin range from 0.19 uM to 14 uM, and oxaliplatin sensitivity is maintained in many cisplatin-resistant cell lines. The KB cell-line used for the present experiments, which grows well as xenografts, exhibits a comparable sensitivity to cisplatin (4 μM) and somewhat less sensitivity to oxaliplatin (IC₅₀=5.4 μM at 72 h) compared with that reported by others. Hence, a single agent efficacy study comparing free oxaliplatin to liposomal oxaliplatin 5a was first conducted in KB xenograft tumors.

Prior to initiation of this study, oxaliplatin was evaluated for drug tolerance in non-tumor bearing immunodeficient mice. Doses above 15 mg/kg (i.e., 20 mg/kg) resulted in dehydration and unacceptable gross body weight losses. The maximum tolerated dose (MTD) for oxaliplatin was determined to be 15 mg/kg in mice, consistent with preclinical data provided the FDA for Eloxatin® approval (NDA 21-492 document). Administration of oxaliplatin at 15 mg/kg in the present studies did not significantly inhibit tumor growth or increase survival compared to the saline control. KB cells used for this experiment exhibited an IC₅₀ of 5.3 uM for oxaliplatin in cytotoxicity testing prior to injection, which is several fold higher than observed for other human tumor cell lines which are partially responsive to oxaliplatin treatment. This may partially explain the inability of oxaliplatin to inhibit tumor growth in this model after a single dose. Although oxaliplatin delayed tumor growth to a size of 0.5 cm³ by five days, this growth inhibitory effect was not maintained over the longer course of the study.

The novel liposomes containing encapsulated oxaliplatin, hereafter referred to as liposomal oxaliplatin 5a, were also dosed once via the same route at dosages of 40 and 60 mg/kg. Unlike free oxaliplatin, a single treatment with liposomal oxaliplatin 5a produced significantly greater tumor growth delay in KB tumors vs. control (P<0.05) (FIG. 8). Both doses of liposomal oxaliplatin 5a tested inhibited tumor growth by 60% compared to saline control. In addition, liposomal oxaliplatin 5a delayed the growth of tumors by 18 and 24 days, (40 and 60 mg/kg, respectively) compared to the saline-treated controls. Moreover, liposomal oxaliplatin 5a treatment also increased median survival between 13 (43%) and 24 days (77%), respectively, vs. saline-treated controls (see, 6/11

FIG. 9 and Table 14) at the two tested dosages. Importantly, only two of ten (20%) liposomal oxaliplatin 5a-treated animals (at the lower dose) were removed for poor health or gross loss of body weight, suggesting that 50 mg/kg is near the MTD for liposomal oxaliplatin 5a.

TABLE 14
Tumor growth inhibition (TGI) and delay (TGD) and
median survival in mice bearing KB xenografts following
treatment with liposomal oxaliplatin 5a or oxaliplatin.

| | TGI | TGD | TGD | Median |
|---|---|---|---|---|
| Treatment and Dose | (%) | (days) | (%) | Survival |
| Control | — | — | — | 31 |
| Oxaliplatin (15 mg/kg) | 0 | 5 | 42 | 25.5 |
| liposomal oxaliplatin 5a (40 | 65 | 18 | 156 | 55 |
| mg/kg) | | | | |
| liposomal oxaliplatin 5a (60 | 63 | 24 | 207 | 44.5 |
| mg/kg) | | | | |

Human colon tumor xenograft models have been widely used to evaluate oxaliplatin-based therapies, primarily using HT29 cells, which are sensitive to low uM concentrations of oxaliplatin in cytotoxicity assays. A number of HT29 cell lines have been generated that are many fold less sensitive to oxaliplatin. The HT29 cell line used in the present studies exhibited a similar sensitivity to oxaliplatin (IC₅₀ 5.4 uM) as reported by others. Two studies of the novel liposome formulation containing oxaliplatin were conducted in this tumor model. In the first study (FIG. 10), liposomal oxaliplatin 5a and Eloxatin were dosed weekly for three weeks. Liposomal oxaliplatin 5a dosed at 22 mg/kg/dose inhibited and delayed tumor growth and increased survival of mice bearing HT29 human colorectal xenograft tumors compared to oxaliplatin dosed at 15 mg/kg/dose on the same schedule (see, 7/11

### FIG. 11, FIG. 12, and Table 15).

TABLE 15
Tumor growth inhibition (TGI) and delay (TGD) and
Survival in mice bearing HT29 xenografts following
treatment with liposomal oxaliplatin 5a or oxaliplatin.

| | TGI | TGD | TGD | Median |
|---|---|---|---|---|
| Treatment and Dose | (%) | (days) | (%) | Survival |
| Control | — | — | — | 29 |
| Eloxatin (15 mg/kg/dose) | 49 | 13 | 93 | 40 |
| liposomal oxaliplatin 5a (22 | 73 | 39 | 279 | 68 |
| mg/kg/dose) | | | | |

In a second study, mice bearing HT-29 colorectal xenografts were treated with liposomal oxaliplatin 5a at 15, 25, or 35 mg/kg/dose weekly for three weeks. Treatment with liposomal oxaliplatin 5a at all dose levels produced smaller tumors than Eloxatin dosed at MTD or saline treatment (8/11

FIG. 13). Eloxatin showed no effect on tumor growth compared to saline, but produced toxicity as seen by loss of body weight and morbidity (see, FIG. 14, 9/11

### FIG. 15, and Table 16).

TABLE 16
Tumor growth inhibition (TGI) and Survival
in mice bearing HT29 xenografts following
treatment with liposomal oxaliplatin 5a or Eloxatin.

| | TGI | Median |
|---|---|---|
| Treatment and Dose | (%) | Survival (Days) |
| Control | — | 37 |
| Eloxatin (15 mg/kg/dose) | 0 | 31 |
| liposomal oxaliplatin 5a | 59 | 38 |
| (15 mg/kg/dose) | | |
| liposomal oxaliplatin 5a | 72 | 44 |
| (25 mg/kg/dose) | | |
| liposomal oxaliplatin 5a | 67 | 45 |
| (35 mg/kg/dose) | | |

A biodistribution and pharmacokinetic study with liposomal oxaliplatin 5a and Eloxatin in immunodeficient mice bearing HT29 xenografts was also conducted. Treatment with liposomes (15 mg/kg oxaliplatin) increased total tumor platinum exposure (AUC) 6 fold greater than treatment with the same dose of Eloxatin (FIG. 16). This observation is consistent with 1) enhanced tumor penetration and retention (EPR effect), and 2) sustained plasma half-life of liposomal oxaliplatin 5a vs. free oxaliplatin, within the circulation and tumor microenvironment. Without wishing to be bound by any particular theory, it is believed that the increased tumor exposure to platinum likely contributed to the increased efficacy observed with liposomes in this HT29 colorectal xenograft model. Plasma platinum levels are shown in 10/11

FIG. 17. Pharmacokinetics and tissue distribution are summarized in Table 17 and Table 18.

TABLE 17
Pharmacokinetics of Eloxatin and liposomal oxaliplatin 5a

| | | liposomal | liposomal |
|---|---|---|---|
| Compound | Eloxatin | oxaliplatin 5a | oxaliplatin 5a |
| Dose (mg/kg)^{a} | 15 | 15 | 45 |
| AUC (hr*ug/ml) | 55 | 5952 | 260623 |
| Cₘₐₓ (μg/ml) | 12 | 141 | 351 |
| CL (ml/hr/kg) | 129 | 1.2 | 0.8 |
| t_{1/2} (hr) | 38 | 17 | 18 |
| Vz (ml/kg) | 7026 | 30 | 22 |

^{a}All doses are given as oxaliplatin molar equivalents.

TABLE 18
Tissue Distribution of Eloxatin and liposomal oxaliplatin 5a

| | | liposomal | liposomal |
|---|---|---|---|
| Compound | Eloxatin | oxaliplatin 5a | oxaliplatin 5a |
| Dose (mg/kg)^{a} | 15 | 15 | 45 |
| Tumor AUC | 127.7 | 821.6 | 1018 |
| (hr*ug/ml) | | | |
| Liver AUC | 347.5 | 750.6 | 772.6 |
| (hr*ug/ml) | | | |
| Spleen AUC | 968.1 | 5319 | 6376 |
| (hr*ug/ml) | | | |
| Kidney AUC | 920.1 | 1369 | 1513 |
| (hr*ug/ml) | | | |
| Muscle AUC | 179.8 | 227.5 | 251.4 |
| (hr*ug/ml) | | | |

^{a}All doses are given as oxaliplatin molar equivalents.
^{b}Samples not measured

Pancreatic ductal adenocarcinomas are highly lethal and resistant to chemotherapy. These tumors are relatively vascular deficient, and have a dense stromal matrix, which is thought to contribute to their resistance to chemotherapeutics. Recently, FOLFIRINOX regimen, which contains oxaliplatin, has shown equivalent or slightly improved efficacy compared to standard of care gemcitabine for first-line treatment in metastatic pancreatic cancer. Favorable activity has been reported in pancreatic cancer with the nanomedicines Abraxane compared to gemcitabine, but treatment options in advanced pancreatic cancer remain very limited. In attempts to model these desmoplastic tumors in xenograft models, researchers have employed selected breast or pancreatic cell lines for evaluation of nanoparticle biodistribution and efficacy studies. In the present studies, low uM IC₅₀ values were observed for Eloxatin in blocking proliferation of BxPC-3 pancreatic cells. Liposomal oxaliplatin 5a was evaluated for single agent multi-dose activity against human BxPC-3 pancreatic adenocarcinoma cells in a xenograft model. Although no significant efficacy was observed using Eloxatin alone in weekly dosing in this model, a significant delay in tumor growth was observed at all tested doses of liposomal oxaliplatin 5a (FIG. 18). Body weight changes and survival rates are shown in 11/11

FIG. 19 and FIG. 20, respectively.

### Combination Agent Efficacy Studies

Liposomal oxaliplatin 5a and several therapeutic agents can be used in preclinical combination studies employing xenograft models to evaluate combination activity in various clinically relevant treatment scenarios, including for example, 5-FU, Cetuximab and gemcitabine.

A combination therapy study using the liposomal oxaliplatin 5a formulation with 5FU (5-fluorouracil) in mice bearing HT29 human colorectal xenografts was also conducted and gave good results.

### Example 6

### Further Evaluation of Liposomal Oxaliplatin Efficacy

The ability of liposomal oxaliplatin to effectively reduce tumor growth on HT29 xenografts was shown to be dependent on the composition of the lipids used in the formulation. Changes in composition resulted in differences in the efficacy and in some instances on the tolerability (toxicity). While relatively fast in vitro oxaliplatin release formulations displayed heightened toxic effects in some comparisons (DMPC vs. DPPC, DSPC) the fast release did not explain differences between POPC and DOPC nor between POPC and DMPC. With the results herein lipids having at least one saturated fatty acid chain on the glycero-phosphatidyl choline were found to be preferred over low cholesterol formulations or formulations containing lipids having sites of unsaturation in both fatty acid chains.

Liposomal formulations that showed efficacy vs. control can be narrowed down to the following set of conditions:
a) a lipid composition that comprises a neutral di-alkyl-glycero-phosphatidyl choline which contains either two saturated fatty acids of carbon length≧C14, or preferably >C14, or one saturated fatty acid and one mono-unsaturated fatty acid with chain lengths ≧C14, or preferably >C14;
b) formulations containing between 25% and 45% (mole %) cholesterol with the above specified neutral di-alkyl-glycero-phosphatidyl cholines displayed efficacy vs. control; and
c) PEGylated liposomes could contain either DSPE-PEG(2000) or DSPE-PEG(5000) as stealth components; however, the use of Cholesterol-PEG(5000) did not demonstrate adequate utility.

The study below illustrates the experiments undertaken to arrive at the beneficial compositions of the invention. Properties evaluated include particle size, drug loading (lipid/oxaliplatin), in vitro release of oxaliplatin, IC50 on HT-29 cell (in vitro), and in vivo efficacy on HT29 tumor xenografts. Lipid compositions include alterations of the fatty acid chain on phosphatidyl cholines, mole % added cholesterol, and various anchors for PEG (long circulating agent).

Experimental:

All liposomal formulations of oxaliplatin were prepared by the following method (EtOH injection with passive loading of oxaliplatin).

### Example 1

### Preparation of E000201-051

| | Amount | |
|---|---|---|
| Material | Used | |
| DSPC(1,2-Distearyl-sn-Glycero-3-Phosphocholine) | 2.80 | g |
| Cholesterol | 1.230 | g |
| DSPE-PEG(2000) [(1,2-distearoyl-sn-glycero-3- | 0.971 | g |
| phosphoethanolamine-N-methoxy(polyethylene | | |
| glycol)-2000] | | |
| Oxaliplatin | 1.6 | g |

Into a 30 mL amber jar was added DSPC, cholesterol, and DSPE-PEG(2000). This lipid mixture was dissolved into 10 mL EtOH (lipids dissolved in EtOH at 65° C.). Into a 250 mL serum bottle was added 1.6 g oxaliplatin (LC labs, 99% FW=397) and 100 mL of aqueous 0.3 M sucrose solution (filtered through 0.2 micron filter prior to use). The oxaliplatin solution was heated to 65° C. in a temperature controlled water bath with magnetic stirring. To the heated solution (completely dissolved oxaliplatin) was added the EtOH solution of lipids giving a milky white suspension. Heating continued at 65° C. for 30 min in the water bath.

The vesicles above were extruded 5× through 0.1 micron double stacked membranes (Whatman, Nuclepore Track-Etched, extrusion carried out in isolator) at 65° C. using a 100 mL Lipex™ extruder under 200-600 psig nitrogen. The resulting liposomes were chilled at 5° C. for 2 days which caused crystallization of excess oxaliplatin. The liposomes were decanted from the crystalline oxaliplatin and were diafiltered against 300 mL 0.3 M sucrose, containing 20 mM acetate buffer, (pH 6.5) using mPES 500 KDa MWCO hallow fibers (KrosFlo Research II model tangential flow diafiltration unit). After 10 volumes of diafiltrate were collected, the liposomal retentate was ultrafiltered to a final volume of ca 30 mLs. The ultrafiltered liposomal material was filtered through 0.2 micron syringe filter (Nylon) into amber serum vials and stored at 5° C.

Particle size and zeta potential were determined (50 uL diluted to 1 mL with normal saline) using a Malvern zeta sizer (DLS) and reported as volume mean values in nm.

Reverse Phase HPLC-ELSD Method for the Identification and Determination of Lipid Components

This is a reversed phase HPLC-ELSD method for the identification and determination of lipid components in liposomal formulations. This method can be applied to formulations and control vehicles undergoing stability studies, in vitro release assays, or in vivo studies.

Procedure:

Prepare two stock solutions of the liposome components in an appropriate organic solvent such as n-propanol or methanol. Ensure the solution is clear and free of crystals. One stock solution is used to prepare a set of calibration standards for lipid quantitation. The other is used to prepare a quality control standard for curve verification. Samples are then diluted in the same organic as the standard curve diluent.

HPLC Conditions:

Column: XSelect CSH C18, 3.5 μm, 3.0×150 mm

Column temperature: 50° C.

Autosampler temperature: 15° C.

Injection volume: 10 μL

Run time: 20 min

ELSD: spray chamber 40° C., drift tube 60° C., RF filter @ 4

Elution Profiles:

Mobile Phase A: 25 mM Ammonium Formate

Mobile Phase B: MeOH:ACN=80:20

Gradient Conditions

| Time | Flow Rate | | |
|---|---|---|---|
| (min) | (mL/min) | % A | % B |
| 0.0 | 1.0 | 15 | 85 |
| 15.0 | 1.0 | 0 | 100 |
| 16.0 | 1.0 | 0 | 100 |
| 16.1 | 1.0 | 15 | 85 |
| 20.0 | 1.0 | 15 | 85 |

Run the calibration curve and QC samples followed by your samples.

Report lipid concentrations in mg/mL as well as the lipid molar ratio.

Quantification of Platinum in Samples by ICP-MS

Reagents: Trace metal grade concentrated nitric acid; Platinum standard; Iridium standard (Ir); QC standard, and Milli-Q water.

Equipment: PerkinElmer Inductively Coupled Plasma Mass Spectrometer (NexION300q ICP-MS).

Determination of Total and Unencapsulated Platinum

The following procedure applies for formulations that are ˜2 mg/mL Platinum: Five hundred microliters of a test formulation is spun through an Amicon, 0.5 mL, 30 kD molecular weight cut-off spin filter (Millipore, cat. #UFC503096) at 9,000 rpm for ten minutes to obtain unencapsulated sample. Ten microliters of the unencapsulated filtrate and original test formulation (for unencapsulated and total Platinum determinations respectively) are each digested in five mLs of concentrated nitric and heated at 70° C., 350 rpm. All samples are then diluted with water to a final dilution of 5,000× for unencapsulated and 200,000× for total in water with Ir internal standard at 2 ng/mL. Samples are ran on the ICP-MS and fit to an eight point linear standard curve ranging from 10-20,000 pg/mL Platinum.

In Vitro Release Assay

Five hundred microliters of the liposomal formulation is loaded into a 100 kD dialysis device, Float-A-Lyzer G2. The loaded Float-A-Lyzer is then inserted into a 50 mL conical tube containing 15 mLs of pH5 PBS, pH7 PBS, or fetal bovine serium (FBS). The tubes are then placed in thermo-mixers set at 37° C., 350 rpm. Samples are taken at 6, 24, and 48 hours for analysis.

After all of the time-points and the “total” sample have been collected, 10 μL of each collected sample is transferred to wells of another 96-deep-well plate, 400 μL of concentrated nitric acid is added to each, and the plate is sealed with a plate-sealer and heated at 70° C., 350 rpm for at least one hour (using a thermo-mixer). Samples are diluted to 200× using water with a final concentration of Ir internal standard of 2 ng/mL. All samples are ran on the ICP-MS and fit to an eight point linear standard curve ranging from 10-20,000 pg/mL Platinum.

Determination of IC50 in HT29 Cells

HT29 human tumor cell line was plated in 96-well tissue culture plates (Costar #3595) at 5×10⁴ cells/mL in a final volume of 0.1 mL of 10% FBS in McCoy's 5a media. All media and growth supplements were obtained from Mediatech (Manassas, Va.). Defined fetal bovine serum was obtained from HyClone (#SH30070.03, lot #AWB96395, Logan, Utah). Plates containing cells were incubated at 37° C. in 5% CO₂ in humidified air for 24 hr. The selected initial cell plating density was chosen based upon the approximated doubling time of the individual human tumor cell line. Test compositions were diluted from above stock solutions to 2.2 mmol/L in Dulbecco's modified phosphate-buffered saline (DPBS; Mediatech, Inc., lot #21031339, Manassas, Va.), then serially diluted three-fold in DPBS to generate a nine point concentration-response curve. 10 uL of diluted test compositions were added to plates in triplicate to achieve the desired final concentrations. Plates containing cells with and without added test compositions were returned to incubation as described above, for a total of 72 hr. For the various treatment times, drug containing media was removed after indicated treatment time and replaced with drug-free media. Subsequently, cell viability was assessed using Alamar Blue. For this purpose, media was removed by pipetting from cultured cells and replaced with 0.1 mL/well of 10% (v/v) Alamar Blue (#BUF012A, AbD Serotec, Raleigh, N.C.) diluted in the appropriate cell culture media. Plates were then returned to incubation as before for appropriate color development, between 2-4 hr. Fluorescence of individual plate wells was measured at 545 nm/590 nm (excitation/emission) using a BioTek Synergy4 microplate reader. Cell viability was calculated as a percentage of measured fluorescence obtained relative to cells treated with culture media alone. IC₅₀ values (μmol/L) were determined with the mean of triplicate values using a Microsoft Excel macro that utilizes nonlinear regression analysis and a four-parameter curve fit model.

| | | | | particle | | | | total | | | % |
|---|---|---|---|---|---|---|---|---|---|---|---|
| | | | Anchor- | size | mol | Chol | mol % | mg/mL | Pt | Lipid/Pt | Free |
| Sample # | MP | PC | PEG | (nm) | % PC | mol % | PEG | lipids | ug/mL | mg/mg | Pt |
| E000201- | MP- | DiC20PC | DSPE- | 96.8 | 48.9% | 46.2% | 4.9% | 72.3 | 1740 | 41.6 | 0.7% |
| 050 | 3772 | | PEG(2000) | | | | | | | | |
| E000201- | MP- | DiC20PC | DSPE- | 90.5 | 60.9% | 34.4% | 4.7% | 60.2 | 1440 | 41.8 | 2.5% |
| 059 | 3773 | | PEG(2000) | | | | | | | | |
| E000201- | MP- | DLPC | DSPE- | 87.5 | 66.0% | 29.2% | 4.8% | 59.9 | 1032 | 58.0 | 3% |
| 010 | 3774 | | PEG(2000) | | | | | | | | |
| E000201- | MP- | DMPC | DSPE- | 89.6 | 50.7% | 44.4% | 4.9% | 58.7 | 1590 | 36.9 | 1.2% |
| 054 | 3758 | | PEG(2000) | | | | | | | | |
| E000201- | MP- | DMPC | DSPE- | 87.6 | 61.0% | 34.2% | 4.8% | 58.0 | 1720 | 33.7 | 0.8% |
| 063 | 3759 | | PEG(2000) | | | | | | | | |
| E000201- | MP- | DMPC | DSPE- | 93 | 65.9% | 28.9% | 5.2% | 56.5 | 1167 | 48.4 | 1% |
| 012 | 3775 | | PEG(2000) | | | | | | | | |
| E000201- | NA | DMPC | DSPE- | 95.6 | 71.2% | 24.0% | 4.8% | 67.9 | 80 | 848.8 | 28% |
| 072 | | | PEG(2000) | | | | | | | | |
| E000201- | MP- | DOPC | DSPE- | 101.2 | 51.1% | 43.6% | 5.2% | 54.2 | 1985 | 27.3 | 2% |
| 026 | 3769 | | PEG(2000) | | | | | | | | |
| E000201- | NA | DOPC | DSPE- | 94.2 | 54.7% | 40.4% | 4.9% | 53.7 | 2258 | 23.8 | 1% |
| 037 | | | PEG(2000) | | | | | | | | |
| E000201- | MP- | DOPC | DSPE- | 95.7 | 65.1% | 29.9% | 5.1% | 54.4 | 1677 | 32.4 | 1% |
| 011 | 3800 | | PEG(2000) | | | | | | | | |
| E000201- | MP- | DOPC | DSPE- | 95.9 | 71.1% | 22.9% | 6.0% | 47.8 | 2256 | 21.2 | 3% |
| 025 | 3771 | | PEG(2000) | | | | | | | | |
| E000201- | MP- | DOPC | DSPE- | 85.3 | 51.9% | 43.3% | 4.8% | 63.8 | 1560 | 40.9 | 0.6% |
| 058 | 3769 | | PEG(2000) | | | | | | | | |
| E000201- | MP- | DOPC | DSPE- | 81.3 | 63.0% | 32.3% | 4.7% | 64.3 | 1540 | 41.8 | 0.7% |
| 067 | 3770 | | PEG(2000) | | | | | | | | |
| E000201- | MP- | DOPC | DSPE- | 88.2 | 71.0% | 24.1% | 4.9% | 59.9 | 1700 | 35.2 | 0.6% |
| 076 | 3771 | | PEG(2000) | | | | | | | | |
| E000201- | MP- | DPetPC | DSPE- | 99.5 | 63.6% | 31.2% | 5.2% | 46.3 | 1805 | 25.7 | 8% |
| 033 | 3777 | | PEG(2000) | | | | | | | | |
| E000201- | MP- | DPetPC | DSPE- | 78.8 | 65.8% | 29.5% | 4.7% | 112.9 | 2223 | 50.8 | 1% |
| 042 | 3777 | | PEG(2000) | | | | | | | | |
| E000201- | MP- | DPoPC | DSPE- | 84.7 | 63.6% | 31.4% | 5.0% | 50.6 | 1366 | 37.0 | 11% |
| 031 | 3792 | | PEG(2000) | | | | | | | | |
| E000201- | MP | DPPC | DSPE- | 83.9 | 58.7% | 35.0% | 6.3% | 71.4 | 1249 | 57.2 | 2% |
| 040 | 3756 | | PEG(2000) | | | | | | | | |
| E000201- | MP- | DPPC | DSPE- | 100.1 | 63.5% | 30.7% | 5.7% | 59.6 | 1240 | 48.1 | 2% |
| 035 | 3779 | | PEG(2000) | | | | | | | | |
| E000201- | MP- | DPPC | DSPE- | 101.2 | 66.3% | 28.8% | 5.0% | 61.4 | 1770 | 34.7 | 2% |
| 013 | 3779 | | PEG(2000) | | | | | | | | |
| E000201- | MP- | DPPC | DSPE- | 103.1 | 72.6% | 21.8% | 5.6% | 52.9 | 893 | 59.2 | 4% |
| 034 | 3778 | | PEG(2000) | | | | | | | | |
| E000201- | MP- | DPPC | DSPE- | 111.4 | 74.4% | 20.7% | 4.9% | 63.7 | 352 | 181.0 | ND |
| 030 | 3778 | | PEG(2000) | | | | | | | | |
| E000201- | MP- | DPPC | DSPE- | 95.5 | 51.7% | 43.6% | 4.7% | 64.2 | 1900 | 33.8 | 1.3% |
| 053 | 3755 | | PEG(2000) | | | | | | | | |
| E000201- | MP | DPPC | DSPE- | 95.3 | 62.8% | 32.3% | 4.9% | 65.7 | 1490 | 44.1 | 1.7% |
| 062 | 3756 | | PEG(2000) | | | | | | | | |
| E000201- | MP- | DPPC | DSPE- | 92.8 | 71.9% | 23.3% | 4.8% | 72.7 | 350 | 207.6 | 7.9% |
| 071 | 3757 | | PEG(2000) | | | | | | | | |
| E000201- | MP- | DSPC | DSPE- | 104 | 65.7% | 29.3% | 5.0% | 50.7 | 1650 | 30.7 | 0% |
| 014 | 3780 | | PEG(2000) | | | | | | | | |
| E000201- | MP- | DSPC | DSPE- | 102.2 | 52.6% | 42.4% | 5.0% | 66.0 | 1830 | 36.0 | 1.0% |
| 051 | 3749 | | PEG(2000) | | | | | | | | |
| E000201- | MP- | DSPC | DSPE- | 97.8 | 62.3% | 33.1% | 4.7% | 61.0 | 1710 | 35.6 | 0.9% |
| 060 | 3750 | | PEG(2000) | | | | | | | | |
| E000201- | MP- | DSPC | DSPE- | 92.4 | 71.7% | 23.5% | 4.8% | 63.3 | 1610 | 39.3 | 2.1% |
| 069 | 3751 | | PEG(2000) | | | | | | | | |
| E000201- | MP- | HSPC | DSPE- | 92.7 | 56.5% | 37.6% | 5.9% | 81.7 | 1841 | 44.4 | 1% |
| 039 | 3799 | | PEG(2000) | | | | | | | | |
| E000201- | MP- | HSPC | DSPE- | 96.7 | 67.0% | 28.1% | 4.9% | 56.4 | 1386 | 40.7 | 2% |
| 015 | 3781 | | PEG(2000) | | | | | | | | |
| E000201- | MP- | HSPC | DSPE- | 96.7 | 50.3% | 44.1% | 5.5% | 56.1 | 1700 | 33.0 | 12% |
| 052 | 3752 | | PEG(2000) | | | | | | | | |
| E000201- | MP- | HSPC | DSPE- | 94.6 | 58.3% | 36.2% | 5.5% | 57.0 | 1700 | 33.5 | 0.7% |
| 061 | 3753 | | PEG(2000) | | | | | | | | |
| E000201- | MP- | HSPC | DSPE- | 86.3 | 67.3% | 27.2% | 5.5% | 55.3 | 1340 | 41.3 | 3.6% |
| 070 | 3754 | | PEG(2000) | | | | | | | | |
| E000201- | MP- | OPPC | DSPE- | 80.5 | 61.5% | 33.3% | 5.2% | 54.6 | 1361 | 40.1 | 11% |
| 032 | 3782 | | PEG(2000) | | | | | | | | |
| E000201- | MP- | POPC | DSPE- | 100.2 | 39.0% | 56.3% | 4.7% | 50.6 | 1513 | 33.4 | 2% |
| 001 | 3788 | | PEG(2000) | | | | | | | | |
| E000201- | MP- | POPC | DSPE- | 103.5 | 41.6% | 53.4% | 5.0% | 74.8 | 2372 | 31.5 | 5% |
| 041 | 3788 | | PEG(2000) | | | | | | | | |
| E000201- | MP- | POPC | DSPE- | 85.2 | 49.9% | 45.3% | 4.8% | 60.2 | 1650 | 36.5 | 1.1% |
| 057 | 3766 | | PEG(2000) | | | | | | | | |
| E000201- | MP- | POPC | DSPE- | 92.2 | 50.2% | 44.6% | 5.2% | 41.7 | 1144 | 36.4 | 3% |
| 002 | 3766 | | PEG(2000) | | | | | | | | |
| E000201- | MP- | POPC | DSPE- | 84.8 | 54.5% | 40.6% | 5.0% | 63.7 | 1685 | 37.8 | 2% |
| 009 | 3747 | | PEG(2000) | | | | | | | | |
| E000201- | MP- | POPC | Cholesterol- | 99.9 | 59.9% | 30.4% | 9.7% | 46.4 | 792 | 58.6 | 8% |
| 023 | 3796 | | PEG(5000) | | | | | | | | |
| E000201- | MP- | POPC | DSPE- | 82 | 60.0% | 35.4% | 4.7% | 59.0 | 1570 | 37.6 | 1.0% |
| 066 | 3767 | | PEG(2000) | | | | | | | | |
| E000201- | MP- | POPC | DSPE- | 92.9 | 64.2% | 28.5% | 7.3% | 57.6 | 1721 | 33.5 | 9% |
| 007 | 3786 | | PEG(2000) | | | | | | | | |
| E000201- | MP- | POPC | DSPE- | 87.6 | 65.2% | 29.6% | 5.1% | 68.4 | 1350 | 50.7 | 0.7% |
| 077 | 3798 | | PEG(5000) | | | | | | | | |
| E000201- | MP- | POPC | DSPE- | 80.7 | 65.3% | 29.6% | 5.1% | 52.7 | 1469 | 35.9 | 4% |
| 008 | 3628 | | PEG(2000) | | | | | | | | |
| E000180- | MP- | POPC | DSPE- | 100 | 66.0% | 29.1% | 4.9% | 87 | 2520 | 35 | 3% |
| 666 | 3628 | | PEG(2000) | | | | | | | | |
| E000201- | MP- | POPC | DSPE- | 85.6 | 66.3% | 31.1% | 2.5% | 33.8 | 833 | 40.5 | 3% |
| 006 | 3787 | | PEG(2000) | | | | | | | | |
| E000201- | MP- | POPC | Cholesterol- | | 66.9% | 28.3% | 4.7% | 70.6 | 1090 | 64.8 | 0.6% |
| 079 | 3796 | | PEG(5000) | | | | | | | | |
| E000201- | MP- | POPC | DSPE- | 79.4 | 69.7% | 25.6% | 4.7% | 62.2 | 1670 | 37.2 | 3.7% |
| 075 | 3768 | | PEG(2000) | | | | | | | | |
| E000201- | MP- | POPC | DSPE- | 80 | 74.6% | 20.7% | 4.7% | 57.2 | 1474 | 38.8 | 15% |
| 003 | 3785 | | PEG(2000) | | | | | | | | |
| E000201- | MP- | POPC | DSPE- | 78.9 | 84.0% | 10.7% | 5.3% | 48.6 | 1034 | 47.0 | 13% |
| 004 | 3784 | | PEG(2000) | | | | | | | | |
| E000201- | MP- | POPC | DSPE- | 74.8 | 95.0% | 0.0 | 5.0 | 58.3 | 1408 | 41.4 | 19% |
| 005 | 3783 | | PEG(2000) | | | | | | | | |
| E000201- | MP- | PSPC | DSPE- | 102.3 | 51.6% | 43.5% | 4.9% | 59.7 | 1810 | 33.0 | 2.8% |
| 055 | 3760 | | PEG(2000) | | | | | | | | |
| E000201- | MP- | PSPC | DSPE- | 6.4 | 62.9% | 32.5% | 4.6% | 52.4 | 1570 | 33.4 | 0.8% |
| 064 | 3761 | | PEG(2000) | | | | | | | | |
| E000201- | MP- | PSPC | DSPE- | 99.4 | 67.3% | 28.1% | 4.6% | 54.0 | 1385 | 39.0 | 3% |
| 017 | 3793 | | PEG(2000) | | | | | | | | |
| E000201- | MP- | PSPC | DSPE- | 90.9 | 70.5% | 24.5% | 5.0% | 58.5 | 920 | 63.6 | 4.7% |
| 073 | 3762 | | PEG(2000) | | | | | | | | |
| E000201- | MP- | SOPC | DSPE- | 92 | 50.4% | 44.9% | 4.8% | 55.0 | 1620 | 34.0 | 1.5% |
| 056 | 3763 | | PEG(2000) | | | | | | | | |
| E000201- | MP- | SOPC | DSPE- | 81.8 | 55.2% | 39.9% | 4.8% | 44.5 | 1069 | 41.6 | 8% |
| 028 | 3791 | | PEG(2000) | | | | | | | | |
| E000201- | MP- | SOPC | DSPE- | 91.8 | 59.4% | 36.2% | 4.4% | 64.6 | 1840 | 35.1 | 1.3% |
| 065 | 3764 | | PEG(2000) | | | | | | | | |
| E000201- | MP- | SOPC | DSPE- | 97.9 | 65.7% | 29.2% | 5.1% | 49.8 | 2209 | 22.5 | 1% |
| 016 | 3790 | | PEG(2000) | | | | | | | | |
| E000201- | MP- | SOPC | DSPE- | 79.2 | 69.8% | 25.7% | 4.5% | 54.5 | 1470 | 37.1 | 0.6% |
| 074 | 3765 | | PEG(2000) | | | | | | | | |
| E000201- | MP- | SOPC | DSPE- | 93.1 | 74.8% | 20.3% | 4.9% | 54.3 | 1971 | 27.5 | 2% |
| 027 | 3789 | | PEG(2000) | | | | | | | | |

| | | | Release | Release | | | |
|---|---|---|---|---|---|---|---|
| | | | 37° C. pH | 37° C. pH | Release | HT29 2 hr | HT29 24 hr |
| | | | 7.4 PBS | 5 PBS | 37° C. | cell | cell |
| Sample # | MP | PC | (48 hr) | (48 hr) | FBS (48 hr) | kill IC50 | kill IC50 |
| E000201-050 | MP-3772 | DiC20PC | 1.1% | 2.4% | 2.4% | >200 | >200 |
| E000201-059 | MP-3773 | DiC20PC | 2.6% | 3.6% | 3.3% | >200 | 89.10 |
| E000201-010 | MP-3774 | DLPC | 32.0% | 33.0% | 94.0% | 26.3 | 1.54 |
| E000201-054 | MP-3758 | DMPC | 1.3% | 2.9% | 1.7% | >200 | >200 |
| E000201-063 | MP-3759 | DMPC | 1.2% | 3.7% | 5.1% | >200 | 79.70 |
| E000201-012 | MP-3775 | DMPC | 3.0% | 4.0% | 8.0% | 213.0 | 32.38 |
| E000201-072 | NA | DMPC | 77.0% | 93.0% | 74.5% | ND | ND |
| E000201-026 | MP-3769 | DOPC | 7.0% | 20.0% | 23.0% | 70.93 | 16.78 |
| E000201-037 | NA | DOPC | NA | NA | NA | ND | ND |
| E000201-011 | MP- | DOPC | 9.0% | 15.0% | 7.0% | 59.8 | 2.87 |
| | 3800 | | | | | | |
| E000201-025 | MP-3771 | DOPC | 9.0% | 25.0% | 99.0% | 56.71 | 3.17 |
| E000201-058 | MP-3769 | DOPC | 4.3% | 15.5% | 6.0% | >200 | 68.22 |
| E000201-067 | MP-3770 | DOPC | 6.5% | 18.0% | 8.6% | 95.35 | 34.69 |
| E000201-076 | MP-3771 | DOPC | 5.6% | 15.5% | 12.6% | 78.39 | 15.73 |
| E000201-033 | MP-3777 | DPetPC | 5.0% | 16.0% | 34.0% | 79.77 | 8.12 |
| E000201-042 | MP-3777 | DPetPC | 11.0% | 18.0% | 12.0% | 69.78 | 16.31 |
| E000201-031 | MP-3792 | DPoPC | 14.0% | 27.0% | 94.0% | 41.40 | 2.67 |
| E000201-040 | MP-3756 | DPPC | 3.0% | 4.0% | 3.0% | >200 | 26.90 |
| E000201-035 | MP-3779 | DPPC | 2.0% | 4.0% | 4.0% | ND | ND |
| E000201-013 | MP-3779 | DPPC | 3.0% | 4.0% | 3.0% | 80.8 | 35.68 |
| E000201-034 | MP-3778 | DPPC | 5.0% | 8.0% | 4.0% | ND | ND |
| E000201-030 | MP-3778 | DPPC | 22.0% | 23.0% | 22.0% | 38.76 | 10.11 |
| E000201-053 | MP-3755 | DPPC | 1.2% | 2.4% | 2.1% | >200 | 86.08 |
| E000201-062 | MP-3756 | DPPC | 1.7% | 2.4% | 1.9% | >200 | 78.94 |
| E000201-071 | MP-3757 | DPPC | 8.3% | 12.6% | 8.9% | >80 | 49.2 |
| E000201-014 | MP-3780 | DSPC | 1.0% | 2.0% | 2.0% | 223.6 | 52.60 |
| E000201-051 | MP-3749 | DSPC | 1.3% | 2.1% | 1.6% | >200 | 91.98 |
| E000201-060 | MP-3750 | DSPC | 1.0% | 1.6% | 1.2% | >200 | 93.70 |
| E000201-069 | MP-3751 | DSPC | 2.0% | 2.6% | 2.4% | >200 | 55.48 |
| E000201-039 | MP-3799 | HSPC | 2.1% | 4.0% | 2.4% | >200 | 35.23 |
| E000201-015 | MP-3781 | HSPC | 1.0% | 3.0% | 2.0% | 90.2 | 44.17 |
| E000201-052 | MP-3752 | HSPC | 0.8% | 1.8% | 1.3% | >200 | 86.65 |
| E000201-061 | MP-3753 | HSPC | 0.9% | 1.4% | 1.0% | >200 | 77.27 |
| E000201-070 | MP-3754 | HSPC | 2.9% | 3.8% | 3.2% | >200 | 46 |
| E000201-032 | MP-3782 | OPPC | 3.0% | 24.0% | 21.0% | 104.84 | 33.01 |
| E000201-001 | MP-3788 | POPC | 2.0% | 3.0% | 4.0% | 84.9 | 30.25 |
| E000201-041 | MP-3788 | POPC | 1.6% | 4.8% | 3.8% | >200 | 52.47 |
| E000201-057 | MP-3766 | POPC | 2.7% | 10.5% | 8.3% | >200 | 56.10 |
| E000201-002 | MP-3766 | POPC | 4.0% | 7.9% | 7.0% | 204.8 | 20.12 |
| E000201-009 | MP-3747 | POPC | 3.0% | 5.0% | 9.0% | 96.5 | 18.90 |
| E000201-023 | MP-3796 | POPC | 4.5% | 11.5% | 7.8% | 58.1 | 7.55 |
| E000201-066 | MP-3767 | POPC | 5.3% | 14.4% | 25.0% | 167 | 27.60 |
| E000201-007 | MP-3786 | POPC | 17.0% | 27.0% | 18.0% | 69.3 | 7.91 |
| E000201-077 | MP-3798 | POPC | 17.1% | 6.7% | 13.0% | >200 | 39.7 |
| E000201-008 | MP-3628 | POPC | 10.0% | 10.0% | 15.0% | 83.1 | 8.70 |
| E000180-666 | MP-3628 | POPC | 5.0% | 4.0% | 8.0% | ND | ND |
| E000201-006 | MP-3787 | POPC | 10.0% | 17.0% | 9.0% | 66.4 | 9.34 |
| E000201-079 | MP-3796 | POPC | 7.8% | 5.0% | 7.4% | ND | ND |
| E000201-075 | MP-3768 | POPC | 8.0% | 20.7% | 31.0% | 77.7 | 15.10 |
| E000201-003 | MP-3785 | POPC | 19.0% | 28.0% | 17.0% | 60.1 | 5.84 |
| E000201-004 | MP-3784 | POPC | 18.0% | 23.0% | 39.0% | 53.7 | 3.18 |
| E000201-005 | MP-3783 | POPC | 27.0% | 32.0% | 28.0% | 36.6 | 2.65 |
| E000201-055 | MP-3760 | PSPC | 1.9% | 2.8% | 2.7% | >200 | 72.1 |
| E000201-064 | MP-3761 | PSPC | 2.0% | 2.9% | 2.5% | >200 | 89.01 |
| E000201-017 | MP-3793 | PSPC | 2.0% | 2.0% | 2.0% | 69.4 | 31.08 |
| E000201-073 | MP-3762 | PSPC | 8.9% | 11.8% | 9.6% | 154 | 19.2 |
| E000201-056 | MP-3763 | SOPC | 2.2% | 8.7% | 5.9% | >200 | 113.34 |
| E000201-028 | MP-3791 | SOPC | 10.0% | 25.0% | 92.0% | 97.80 | 25.92 |
| E000201-065 | MP-3764 | SOPC | 4.2% | 9.6% | 7.4% | >200 | 94.20 |
| E000201-016 | MP-3790 | SOPC | 7.0% | 11.0% | 57.0% | 221.4 | 22.32 |
| E000201-074 | MP-3765 | SOPC | 5.0% | 14.1% | 8.9% | >200 | 60.21 |
| E000201-027 | MP-3789 | SOPC | 12.0% | 26.0% | 57.0% | 92.36 | 22.44 |

### Example 7

### Further Evaluation of Liposomal Oxaliplatin Efficacy In Vivo Studies

Liposomal oxaliplatin formulations with variable liposome compositions were evaluated for tolerance in mice and efficacy in mice bearing HT29 human colorectal xenograft tumors.

Study Design

Acute mouse tolerance assay. Female Hsd:Athymic Nude-FoxN1 nu/mu mice were given a single intravenous (IV) dose of test article at 30, 36 or 45 mg/kg. All doses were given as oxaliplatin equivalent doses. Mice were monitored and weighed for 14 days following injection. Mice found moribund or who have lost greater than 20% body weight were removed from the study.

Mouse xenograft efficacy study. Female Hsd:Athymic Nude-FoxN1 nu/mu mice were each implanted with 2.5×10⁶ HT29 human colorectal cells subcutaneous into the right flank. Once tumors reached a median volume of 200 mm³, 50 animals were randomized and normalized by tumor volume into treatment groups. Animals without tumors were not included in the study. Each animal was given a single intravenous (IV) dose of liposomal oxaliplatin formulation test article, Eloxatin positive control article or saline each week for three weeks (q7d×3). Test articles were given as oxaliplatin equivalent doses.

Measures and Statistics

Tumor volume was determined using a tumor imaging system (Biopticon) 2-3 times per week. Body weights were measured weekly. Tumor volume data was analyzed to determine the ratio of treated versus control tumor volumes (% T/C). Mice were removed from the study if they lost 20% of their initial bodyweight, became moribund, or if their tumor volume exceeded 2500 mm³ or ulcerated. If less than half of the initial cohort of mice remained, that group was no longer included in further tumor analysis.

Ratio of Treated versus Control Tumor volume (% T/C) was calculated on the last day the control group had at least half the original animals remaining on study. % T/C calculated by:

100×(mean tumor volume treatment group)/(mean tumor volume control group).

Statistical comparison of the treatment groups for tumor growth employed a one-way ANOVA comparison on the mean measurements of each dose group at the last time half or more of saline treated mice remained on study and again just prior to the removal of additional groups for falling below 50% of mice on study. Where statistical significance (p<0.05) was observed, a Newman-Keuls post hoc comparison test was conducted. All statistical analyses were conducted using GraphPad Prism 6, and criterion for statistical significance was set at p<0.05.

Results

Liposomal oxaliplatin formulations in Table 1 were evaluated for tolerance with a single intravenous dose of, 30, 36, or 45 mg/kg. Five formulations exhibited signs of severe toxicity which included body weight losses greater than 20% or morbidity. The five remaining formulations, in Table 1, tolerated 45 mg/kg dose of liposomal oxaliplatin without signs of severe toxicity.

Liposomal oxaliplatin formulations (25 mg/kg), Eloxatin (10, 15 mg/kg), and saline were administered to mice bearing HT29 human xenograft tumors weekly for three weeks. Six liposomal oxaliplatin formulations shown in Table 2 produced severe toxicity, while twenty-five additional formulations shown in Table 3 tolerated this level of dosing. Twenty-four of the twenty-five tolerated formulations produced efficacy with tumor volumes significantly smaller than tumors from saline treated mice (p<0.05). These twenty-four liposomal oxaliplatin formulations inhibited tumor growth, producing treatment to control tumor volume ratios (% T/C) ranging from 25% (most efficacious) to 58% (least efficacious). One tolerated liposomal oxaliplatin formulation (MP-3796) did not inhibit tumor growth significantly compared to saline treatment and produced a % T/C of 81%. Administration of three weekly IV doses of 10 or 15 mg/kg Eloxatin, the comparator cytotoxic agent, significantly inhibited tumor growth compared to saline treatment (p<0.05) in only one of four studies and produced % T/C ranging from 53% to 88%.

TABLE 1
Acute mouse tolerance assay

| Study | | | | Dose | |
|---|---|---|---|---|---|
| Number | Formulation | Lot No. | Composition | (Single) | Response |
| E-000217- | MP-3796 | E-000201-023 | POPC:Chol:Chol- | 36 mg/kg | Severe Toxic |
| 007 | | | PEG(2000) (65:30:5) | | Dose |
| E-000217- | MP-3770 | E-000201-011 | DOPC:Chol:DSPE- | 45 mg/kg | Severe Toxic |
| 007 | | | PEG(2000) (65:30:5) | | Dose |
| E-000217- | MP-3774 | E-000201-010 | DLPC:Chol:DSPE- | 45 mg/kg | Severe Toxic |
| 007 | | | PEG(2000) (65:30:5) | | Dose |
| E-000217- | MP-3783 | E-000201-005 | POPC:Chol:DSPE- | 45 mg/kg | Severe Toxic |
| 007 | | | PEG(2000) (95:0:5) | | Dose |
| E-000217- | N/A | E-000201-037 | DOPC:Chol:DSPE- | 30 mg/kg | Severe Toxic |
| 009 | | | PEG(2000) (55:40:5) | | Dose |
| E-000217- | MP-3777 | E-000201-033 | DPetPC:Chol:DSPE- | 45 mg/kg | Tolerated |
| 007 | | | PEG(2000) (65:30:5) | | Dose |
| E-000217- | MP-3781 | E-000201-015 | HSPC:Chol:DSPE- | 45 mg/kg | Tolerated |
| 007 | | | PEG(2000) (65:30:5) | | Dose |
| E-000217- | MP-3628 | E-000201-008 | POPC:Chol:DSPE- | 45 mg/kg | Tolerated |
| 007 | | | PEG(2000) (65:30:5) | | Dose |
| E-000217- | MP-3788 | E-000201-001 | POPC:Chol:DSPE- | 45 mg/kg | Tolerated |
| 007 | | | PEG(2000) (40:55:5) | | Dose |
| E-000217- | MP-3756 | E-000201-013 | DPPC:Chol:DSPE- | 45 mg/kg | Tolerated |
| 009 | | | PEG(2000) (65:30:5) | | Dose |

TABLE 2
Formulations with severe toxicity after multiple doses in tumor bearing mice

| Study | Formulation | | | Dose | |
|---|---|---|---|---|---|
| Number | No. | Lot No. | Composition | (Weekly ×3) | Response |
| E-000254- | MP-3777 | E-000201-042 | DPetPC:Chol:DSPE- | 25 mg/kg | Severe Toxic |
| 003 | | | PEG(2000) (65:30:5) | | Dose |
| E-000266- | MP-3770 | E-000201-067 | DOPC:Chol:DSPE- | 25 mg/kg | Severe Toxic |
| 015 | | | PEG(2000) (60:35:5) | | Dose |
| E-000266- | MP-3771 | E-000201-076 | DOPC:Chol:DSPE- | 25 mg/kg | Severe Toxic |
| 015 | | | PEG(2000) (70:25:5) | | Dose |
| E-000297- | MP-3758 | E-000201-054 | DMPC:Chol:DSPE- | 25 mg/kg | Severe Toxic |
| 001 | | | PEG(2000) (50:45:5) | | Dose |
| E-000297- | MP-3769 | E-000201-058 | DOPC:Chol:DSPE- | 25 mg/kg | Severe Toxic |
| 001 | | | PEG(2000) (50:45:5) | | Dose |
| E-000297- | MP-3759 | E-000201-063 | DMPC:Chol:DSPE- | 25 mg/kg | Severe Toxic |
| 001 | | | PEG(2000) (60:35:5) | | Dose |

TABLE 3

| | | | | Dose | | |
|---|---|---|---|---|---|---|
| Study | Formulation | | | (Weekly | | |
| Number | No. | Lot No. | Composition | X3) | % T/C | Significance^{a} |
| E- | MP-3772 | E-000201- | DiC20PC:Chol:DSPE- | 25 mg/kg | 25 | P < 0.05 |
| 000297- | | 050 | PEG(2000) (50:45:5) | | | |
| 001 | | | | | | |
| E- | MP-3766 | E-000201- | POPC:Chol:DSPE- | 25 mg/kg | 28 | P < 0.05 |
| 000297- | | 057 | PEG(2000) (50:45:5) | | | |
| 001 | | | | | | |
| E- | MP-3767 | E-000201- | POPC:Chol:DSPE- | 25 mg/kg | 28 | P < 0.05 |
| 000266- | | 066 | PEG(2000) (60:35:5) | | | |
| 015 | | | | | | |
| E- | MP-3760 | E-000201- | PSPC:Chol:DSPE- | 25 mg/kg | 29 | P < 0.05 |
| 000297- | | 055 | PEG(2000) (50:45:5) | | | |
| 001 | | | | | | |
| E- | MP-3763 | E-000201- | SOPC:Chol:DSPE- | 25 mg/kg | 29 | P < 0.05 |
| 000297- | | 056 | PEG(2000) (50:45:5) | | | |
| 001 | | | | | | |
| E- | MP-3764 | E-000201- | SOPC:Chol:DSPE- | 25 mg/kg | 29 | P < 0.05 |
| 000266- | | 065 | PEG(2000) (60:35:5) | | | |
| 015 | | | | | | |
| E- | MP-3773 | E-000201- | DiC20PC:Chol:DSPE- | 25 mg/kg | 30 | P < 0.05 |
| 000297- | | 059 | PEG(2000) (60:35:5) | | | |
| 001 | | | | | | |
| E- | MP-3768 | E-000201- | POPC:Chol:DSPE- | 25 mg/kg | 31 | P < 0.05 |
| 000266- | | 075 | PEG(2000) (70:25:5) | | | |
| 015 | | | | | | |
| E- | MP-3761 | E-000201- | PSPC:Chol:DSPE- | 25 mg/kg | 33 | P < 0.05 |
| 000297- | | 064 | PEG(2000) (60:35:5) | | | |
| 001 | | | | | | |
| E- | MP-3765 | E-000201- | SOPC:Chol:DSPE- | 25 mg/kg | 35 | P < 0.05 |
| 000266- | | 074 | PEG(2000) (70:25:5) | | | |
| 015 | | | | | | |
| E- | MP-3762 | E-000201- | PSPC:Chol:DSPE- | 25 mg/kg | 35 | P < 0.05 |
| 000266- | | 073 | PEG(2000) (70:25:5) | | | |
| 015 | | | | | | |
| E- | MP-3788 | E-000201- | POPC:Chol:DSPE- | 25 mg/kg | 39 | P < 0.05 |
| 000254- | | 041 | PEG(2000) (40:55:5) | | | |
| 003 | | | | | | |
| E- | MP-3753 | E-000201- | HSPC:Chol:DSPE- | 25 mg/kg | 44 | P < 0.05 |
| 000217- | | 061 | PEG(2000) (60:35:5) | | | |
| 016 | | | | | | |
| E- | MP-3750 | E-000201- | DSPC:Chol:DSPE- | 25 mg/kg | 45 | P < 0.05 |
| 000217- | | 060 | PEG(2000) (60:35:5) | | | |
| 016 | | | | | | |
| E- | MP-3754 | E-000201- | HSPC:Chol:DSPE- | 25 mg/kg | 45 | P < 0.05 |
| 000217- | | 070 | PEG(2000) (70:25:5) | | | |
| 016 | | | | | | |
| E- | MP-3798 | E-000201- | POPC:Chol:DSPE- | 25 mg/kg | 46 | P < 0.05 |
| 000266- | | 077 | PEG(5000) (65:30:5) | | | |
| 015 | | | | | | |
| E- | MP-3628 | E-000180- | POPC:Chol:DSPE- | 25 mg/kg | 46 | P < 0.05 |
| 000217- | | 666 | PEG(2000) (65:30:5) | | | |
| 016 | | | | | | |
| E- | MP-3752 | E-000201- | HSPC:Chol:DSPE- | 25 mg/kg | 47 | P < 0.05 |
| 000217- | | 052 | PEG(2000) (50:45:5) | | | |
| 016 | | | | | | |
| E- | MP-3755 | E-000201- | DPPC:Chol:DSPE- | 25 mg/kg | 48 | P < 0.05 |
| 000217- | | 053 | PEG(2000) (50:45:5) | | | |
| 016 | | | | | | |
| E- | MP-3756 | E-000201- | DPPC:Chol:DSPE- | 25 mg/kg | 53 | P < 0.05 |
| 000254- | | 040 | PEG(2000) (65:30:5) | | | |
| 003 | | | | | | |
| E- | Eloxatin | DF135D | Oxaliplatin | 10 mg/kg | 53 | P < 0.05 |
| 000266- | | | | | | |
| 015 | | | | | | |
| E- | MP-3799 | E-000201- | HSPC:Chol:DSPE- | 25 mg/kg | 54 | P < 0.05 |
| 000254- | | 039 | PEG(2000) (55:40:5) | | | |
| 003 | | | | | | |
| E- | MP-3749 | E-000201- | DSPC:Chol:DSPE- | 25 mg/kg | 55 | P < 0.05 |
| 000217- | | 051 | PEG(2000) (50:45:5) | | | |
| 016 | | | | | | |
| E- | MP-3756 | E-000201- | DPPC:Chol:DSPE- | 25 mg/kg | 56 | P < 0.05 |
| 000217- | | 062 | PEG(2000) (60:35:5) | | | |
| 016 | | | | | | |
| E- | MP-3751 | E-000201- | DSPC:Chol:DSPE- | 25 mg/kg | 58 | P < 0.05 |
| 000217- | | 069 | PEG(2000) (70:25:5) | | | |
| 016 | | | | | | |
| E- | Eloxatin | DF135D | Oxaliplatin | 10 mg/kg | 69 | ns |
| 000297- | | | | | | |
| 001 | | | | | | |
| E- | Eloxatin | DF135D | Oxaliplatin | 15 mg/kg | 81 | ns |
| 000217- | | | | | | |
| 016 | | | | | | |
| E- | MP-3796 | E-000201- | POPC:Chol:Chol- | 25 mg/kg | 81 | ns |
| 000266- | | 079 | PEG(5000) (65:30:5) | | | |
| 015 | | | | | | |
| E- | Eloxatin | CL77D | Oxaliplatin | 10 mg/kg | 88 | Ns |
| 000254- | | | | | | |
| 003 | | | | | | |

^{a}Statistical comparison to mean tumor volume of saline treated mice, One-way ANOVA, Neuman-Keuls post-hoc test. ns = non-significant.

Liposomal oxaliplatin formulations were prepared using the EtOH dilution method which is inherently a “passive” encapsulation method. Formulations which satisfy several characteristics were desirable as potential therapeutic, injectable materials. For parenteral applications, a key consideration was the particle size of the formed vesicles. Particle sizes for parenteral liposomal formulations have been found to be optimal in the 80-120 nm range. Greater particle size has been reported to lead to greater uptake by the reticular endothelial system (RES) while smaller particles tend to be less stable toward release of encapsulated material. Therefore an initial criterion in our selection process was the generation of liposomal particles with a volume mean size between 80-120 nm.

The encapsulation of oxaliplatin within the aqueous interior of the vesicles was dependent on the concentration of lipids in EtOH and on the concentration of oxaliplatin in the aqueous phase during the vesicle forming process. The ability of the vesicles to retain the oxaliplatin during processing was crucial in obtaining the greatest loading efficiency. Formulations were analyzed for lipid concentration, total oxaliplatin content and unencapsulated oxaliplatin upon completion of processing. Those formulations which gave lipid to oxaliplatin ratios between 20 and 100 were regarded as acceptable for further evaluations. Those that gave higher ratios indicated that retention of oxaliplatin was severely limited during processing and the obtained material, therefore, contained a concentration of oxaliplatin which was not deemed sufficient for efficacious use. Formulations which, upon analysis, contained a high level of unencapsulated oxaliplatin were excluded, as these formulations were inherently unstable toward oxaliplatin release in storage. In general, those formulations with di-alkyl-glycero-phosphatidyl choline containing fatty acids with <C14 chain lengths or which contained low amounts of cholesterol did not encapsulate sufficient amounts of oxaliplatin.

Oxaliplatin formulations which possessed drug to lipid ratios of 20-100 and were between 80-120 nm in size were evaluated for oxaliplatin release in vitro. The in vitro release method tested the thermal stability (37° C.) of the vesicles and formulations were evaluated at two pH values (5 and 7.4). A high release of oxaliplatin was indicative of the inability of the liposome to retain oxaliplatin and excessive release was an indication of poor stability. During the course of our testing of these oxaliplatin formulations, we observed that in several cases a large (greater than 2×) difference in release of oxaliplatin occurred at the lower pH (5). The greatest difference was observed with compositions containing at least one unsaturated fatty acid in the di-alkyl-glycero-phosphatidyl choline component. This pH sensitivity was not anticipated; however, it was not clear as the reason for this observation nor was it obvious that this has any in vivo effect on performance. Many of the formulations prepared displayed rather slow release of oxaliplatin (<5% over 48 hr at 37° C.). Those with slow release were regarded as potential formulations that maintain a constant low level of unencapsulated oxaliplatin in circulation and which might display minimal toxicity. Those formulations with low release may, however, not provide for adequate bioavailability of oxaliplatin in vivo and may show minimal efficacy.

In addition to the in vitro release studies at pH 5 and 7.4, release of oxaliplatin was evaluated in the presence of serum proteins and other biological material at 37° C. in a 90% fetal bovine serum (FBS) matrix. This release measurement was intended to mimic potential release in vivo. Rapid or burst release of oxaliplatin in FBS would indicate the inability of the vesicles to maintain oxaliplatin delivery over an extended period of time while in circulation, although, this was not specifically shown to be directly correlated with in vivo oxaliplatin release into circulation. The occurrence of a fast release of oxaliplatin in vitro (FBS media) was taken as a negative aspect of the formulation and those formulations may have led to more problematic toxicity in subsequent in vivo studies. Those formulations that displayed slow release (<5% over 48 hr at 37° C.) in vitro were regarded as potentially low toxicity materials; however, it was not clear as to whether the release rate (as observed in vitro) would provide an adequate amount of available oxaliplatin for efficacy (as measured by tumor growth suppression in vivo). In general, it was observed that the release of oxaliplatin increased with shorter chain fatty acid (DLPC) and with a lower molar % of cholesterol (based on total lipids). For di-alkyl-glycero-phosphatidyl cholines containing fatty acids of equal chain length, those with unsaturation displayed greater release rates of oxaliplatin than the fully saturated counterparts.

Formulations of oxaliplatin which provided vesicles of volume mean particle size between 80-120 nm, encapsulation ratio of less than 100 (lipid to oxaliplatin) and displayed an in vitro release of <25% over 48 hrs were considered for in vivo studies. Oxaliplatin formulations, to be considered as potential drug products must satisfy two important criteria:
1) The formulated oxaliplatin must retain the ability to suppress tumor growth and compare favorably with, Eloxatin; and
2) The formulated oxaliplatin should not display additional toxicity to Eloxatin, and preferably it should display a benefit toward patient safety over Eloxatin.

The formulations which met the in vitro criteria as described above were evaluated in vivo using an HT29 human colorectal xenograft tumor model in mice.

Formulations which caused death or significant weight loss at the specified dose of 45 mg/kg (single dose) or 25 mg/kg (3-weakly doses) were considered toxic. These formulations included the low cholesterol, short chain formulation (≦C14) and all of the formulations containing either DOPC or DiPetPC. Both formulations contain di-alkyl-glycero-phosphatidyl cholines with both fatty acid chains containing unsaturation. The toxic nature of these formulations was not well understood and was not predictable based on their in vitro characteristics. In fact, the release rates observed in vitro and the IC50 values obtained were virtually identical to formulations containing POPC (single chain containing unsaturation), which were tolerated in these studies.

All of the formulations which displayed slow in vitro release were acceptable with little to no weight loss after 3 weekly doses at 25 mg/kg. This included all of the formulations containing di-alkylphophstidylcholines containing saturated fatty acid chains of greater than C14 and which contain at least 25% (mole %) cholesterol. The formulations containing POPC and SOPC (with one saturated fatty acid and one unsaturated fatty acid) also gave good results with little to no weight loss/toxicity at 3 weekly doses at 25 mg/kg. While this dosing regimen was tolerated, the maximum tolerated dose of these formulations was not determined and may be above 25 mg/kg for 3 weekly doses. Reduction of dose may still provide efficacy without toxic events with those formulations that gave rise to unacceptable toxicity at 25 mg/kg (3 weekly doses). Eloxatin, the commercial formulation of oxaliplatin was determined to have an MTD between 10 and 15 mg/kg (3 weekly doses). As judged from the above results, all of the formulations containing at least one saturated fatty acid and contain chains of greater than C14 along with at a minimum of 25% by weight cholesterol were able to achieve oxaliplatin equivalent dosing levels at 167% that of Eloxatin.

Formulations of oxaliplatin that satisfied the in vitro criteria as acceptable were evaluated for efficacy in the HT29 human colorectal xenograft tumor model in mice. Included as comparison in each study group were saline as control and Eloxatin (as current gold standard). Those formulations which displayed efficacy (as judged by % T/C; tumor volume ratio of treated vs. saline control) included all formulations which were shown to have a safety profile greater than Eloxatin as long as the PEG containing moiety contained DSPE. The formulation containing a cholesterol anchored PEG did not show efficacy in this model. Differentiation from Eloxatin, although not statistically significant in all cases, was shown for all formulations except for the cholesterol anchored PEG formulation and those formulations which displayed unacceptable tolerance to the 3 weekly doses at 25 mg/kg. It was tempting to conclude that the formulations that contain PC moieties with both alkyl chains saturated gave higher % T/C than those that contain one chain with unsaturation (POPC and SOPC). However, this was only a trend that was not verified with statistical significance. The one exception to the trend was in the case of the PC containing saturated C20 chains. The greater efficacy (lower % T/C) with this PC was not expected nor was it predictable based on any in vitro studies or from any of its physical/chemical properties.

It is assumed that the release of oxaliplatin from the vesicle was a necessity for biological activity and that the rate of release plays an important part in tumor growth suppression. The diverse range of oxaliplatin release rates observed in vitro for the formulations described above raise important questions as to the relevance of the in vitro release to in vivo activity. Formulations giving release rates as high as 20% in vitro (POPC containing) and those as low as 1-2% (DSPC, HSPC) all inhibited tumor growth and gave % T/C lower than (or from a statistical analysis equal to) Eloxatin without causing morbidity or adverse weight reduction in the mice. How these formulations gave rise to efficacy as a function of in vivo oxaliplatin release has not been determined. As outlined previously, the release of oxaliplatin from the POPC:cholesterol:DSPE-PEG(2000) formulation (MP-3628) has been studied extensively in vivo and has demonstrated an extended release profile of oxaliplatin in circulation. Accordingly, a set of formulations is provided herein that are more tolerable than Eloxatin while maintaining at least equivalent, if not greater, efficacy than Eloxatin. This set of formulations includes:
1) PC with at a least one saturated fatty acid chain that is greater than C14 in length
2) PC with both saturated fatty acid chains of greater than C14 in length
3) PC with one fatty acid chain containing one unsaturated bond and is greater than C14 in length
4) Cholesterol in the formulation which contains at least 25 mole %
5) A PEGylated PC at less than 7.5 mole %

Surprisingly, the use of DOPC at all cholesterol levels appeared to cause greater toxicity than the corresponding POPC series. It was also surprising that the use of formulations with slow in vitro release profiles (i.e., DSPC and HSPC) would display efficacy, especially the unusual high efficacy (low % T/C) seen with the di C20 PC formulations.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

1. A composition for the treatment of cancer, comprising:
(a) zwitterionic liposomes consisting essentially of from about 50 mol % to about 70 mol % of a phosphatidylcholine lipid or mixture of phosphatidylcholine lipids, from about 25 mol % to about 45 mol % of cholesterol, and from about 2 mol % to about 8 mol % of a PEG-lipid; and
(b) oxaliplatin, encapsulated in said liposome in an amount such that the ratio of the total lipid weight to the oxaliplatin weight is from about 20:1 to about 65:1;
wherein said phosphatidylcholine lipid or mixture of phosphatidylcholine lipids have fatty acid chains of 14 carbon atoms or more, and no more than one of the two fatty acid chains is unsaturated.

2. A composition of claim 1, wherein oxaliplatin is encapsulated in said liposome in an amount such that the ratio of the total lipid weight to the oxaliplatin weight is from about 30:1 to about 45:1.

3. A composition of claim 1, wherein said phosphatidyl choline lipid or mixture of phosphatidylcholine lipids is other than hydrogenated soy phosphatidylcholine (HSPC) or other than a mixture comprising HSPC.

4. A composition of claim 1, wherein said phosphatidylcholine lipid or mixture of phosphatidylcholine lipids have fatty acid chains of 15 carbon atoms or more.

5. A composition of claim 1, wherein said phosphatidylcholine lipid is selected from the group consisting of palmitoyloleoylphosphatidylcholine (POPC), distearoylphosphatidylcholine (DSPC), stearoyloleoylphosphatidylcholine (SOPC), and dipalmitoylphosphatidylcholine (DPPC).

6. A composition of claim 1, wherein said phosphatidylcholine lipid is POPC.

7. A composition of claim 1, wherein the PEG-lipid is a diacyl-phosphatidylethanolamine-N-[methoxy(polyethene glycol)].

8. A composition of claim 1, wherein the PEG-lipid is selected from the group consisting of distearoyl-phosphatidylethanolamine-N-[methoxy(polyethene glycol)-2000] (DSPE-PEG-2000) and distearoyl-phosphatidylethanolamine-N-[methoxy(polyethene glycol)-5000] (DSPE-PEG-5000).

9. A composition of claim 1, wherein the zwitterionic liposome comprises about 55 mol % POPC, about 40 mol % cholesterol, and about 5 mol % DSPE-PEG(2000).

10. A composition of claim 1, wherein the zwitterionic liposome comprises about 65 mol % POPC, about 30 mol % cholesterol, and about 5 mol % DSPE-PEG(2000).

11. A composition of claim 9, wherein the ratio of the total lipid weight to the oxaliplatin weight is about 50:1.

12. A composition of claim 9, wherein the ratio of the total lipid weight to the oxaliplatin weight is from about 30:1 to about 35:1.

13. A composition of claim 1, wherein said zwitterionic liposomes have an average particle size of from about 75 to about 125 nm.

14. A composition of claim 1, wherein said zwitterionic liposomes have an average particle size of about 90 nm.

15. A composition of claim 1, wherein said zwitterionic liposomes are prepared by a method comprising:
a) forming a lipid solution comprising the phosphatidylcholine lipid, the cholesterol, the PEG-lipid, and a solvent selected from the group consisting of a C₁₋₄alkanol and a C₁₋₄alkanol/water mixture;
b) mixing the lipid solution with an aqueous buffer to form multi-lamellar vesicles (MLVs); and
c) extruding the MLVs through a porous filter to form small unilamellar vesicles (SUVs);
d) diafiltrating to remove un-encapsulated oxaliplatin from the liposomal formulation;
thereby forming said zwitterionic liposomes.

16. A composition of claim 15, wherein encapsulation of the oxaliplatin is conducted by including the oxaliplatin in the aqueous buffer during formation of the MLVs.

17. A composition of claim 15, wherein the method further comprises;
e) sterile filtering said zwitterionic liposomes.

18. A method of treating cancer, said method comprising administering to a subject in need thereof a composition of claim 1.

19. A method of claim 18, wherein said cancer is a solid tumor cancer selected from the group consisting of bladder cancer, colorectal cancer, gastric cancer, esophageal cancer, non-small cell lung cancer, pancreatic cancer, breast cancer, ovarian cancer and prostate cancer.

20. A method of claim 18, wherein said composition comprises:
a) zwitterionic liposomes consisting essentially of about 55 mol % POPC, about 40 mol % cholesterol, and about 5 mol % DSPE-PEG(2000); and
b) oxaliplatin, encapsulated in said liposome in an amount such that the ratio of the total lipid weight to the oxaliplatin weight is about 50:1.

21. A composition of claim 1, wherein said zwitterionic liposomes are liposomes selected from the group consisting of HSPC/Chol/DSPE-PEG(2000), 65/30/5; POPC/Chol/DSPE-PEG(2000), 65/30/5; and DPPC/Chol/DSPE-PEG(2000), 65/30/5 liposomes.

22. A composition of claim 1, wherein said zwitterionic liposomes are liposomes selected from the group consisting of POPC/Chol/DSPE-PEG(2000), 50/45/5; PSPC/Chol/DSPE-PEG(2000), 50/45/5; DiC20PC/Chol/DSPE-PEG(2000), 50/45/5; HSPC/Chol/DSPE-PEG(2000), 50/45/5; DPPC/Chol/DSPE-PEG(2000), 50/45/5; DSPC/Chol/DSPE-PEG(2000), 50/45/5; and SOPC/Chol/DSPE-PEG(2000), 50/45/5 liposomes.

23. A composition of claim 1, wherein said zwitterionic liposomes are liposomes selected from the group consisting of POPC/Chol/DSPE-PEG(2000), 70/25/5; PSPC/Chol/DSPE-PEG(2000), 70/25/5; HSPC/Chol/DSPE-PEG(2000), 70/25/5; DSPC/Chol/DSPE-PEG(2000), 70/25/5; and SOPC/Chol/DSPE-PEG(2000), 70/25/5 liposomes.

24. A composition of claim 1, wherein said zwitterionic liposomes are liposomes selected from the group consisting of POPC/Chol/DSPE-PEG(2000), 60/35/5; PSPC/Chol/DSPE-PEG(2000), 60/35/5; HSPC/Chol/DSPE-PEG(2000), 60/35/5; DSPC/Chol/DSPE-PEG(2000), 60/35/5; DPPC/Chol/DSPE-PEG(2000), 60/35/5; DiC20PC/Chol/DSPE-PEG(2000), 60/35/5; and SOPC/Chol/DSPE-PEG(2000), 60/35/5 liposomes.

25. A composition of claim 1, wherein said zwitterionic liposomes are POPC/Chol/DSPE-PEG(5000), 65/30/5 liposomes.
